(19) United States
(12) Patent Application Publication
(10) Pub. No.: US 2011/0076278 A1
(43) Pub. Date: Mar. 31, 2011

(54) Modulators of Hypoxia Inducible Factor-1 and Related Uses for the Treatment of Ocular Disorders

(76) Inventor: Mehran Khodadoust, Brookline, MA (US)

(21) Appl. No.: 11/989,362

(22) PCT Filed: Aug. 1, 2006
(86) PCT No.: PCT/US2006/030224

§ 371 Date: Aug. 29, 2008

Related U.S. Application Data
(60) Provisional application No. 60/704,795, filed on Aug. 2, 2005.

(51) Int. Cl. A61K 31/7048; A61K 39/395; A61K 31/58; A61P 27/02; A61P 27/06; A61P 29/00; A61P 37/06

(52) U.S. Cl. 424/158.1; 514/26; 514/171; 514/172

(57) ABSTRACT

Methods for treatment of ocular disorders using steroids modulate the effects of local and systemic hypoxic events mediated by hypoxia inducible factor-1 (HIF-1). Steroids that are useful as HIF-1 modulators include bufalin, digitoxigenin, digoxin, lanatoside C, strophantin K, uzarigenin, ouabain and proscillaridin. In some embodiments the ocular disorder is characterized by ischmia.

### BACKGROUND OF THE INVENTION

Hypoxia provokes a wide range of physiological and cellular responses in humans and other mammals The effects of hypoxia vary qualitatively depending on the length of time over which hypoxic conditions are maintained. Acute hypoxia is characterized by increased respiratory ventilation, but after 3-5 minutes, ventilation declines. Individuals exposed to chronic hypoxic conditions undergo a suite of responses including decreased heart rate and increased blood pressure. Metabolically, hypoxia causes decreased glucose oxidation with a shift from oxidative phosphorylation to glycolysis. Glycolysis provides a poorer yield of energy from carbohydrates, and oxidation of fatty acids is greatly reduced. Perhaps for these reasons, hypoxia also triggers increased consumption of carbohydrates. Hypoxia stimulates production of erythropoietin, which in turn leads to an increase in the red blood cell count. Hypoxia may occur at the level of the whole organism, as, for example, when ventilation is interrupted or when oxygen availability is low. Hypoxia may also occur at a local level essentially any time oxygen consumption outpaces the supply from the bloodstream. Ischemic events are severe forms of local hypoxia that lead to cell death. Recent discoveries relating to the transcription factor have provided considerable insight into the local, cellular response to hypoxia, but our understanding of how the overall physiological response is regulated, and how the systemic and local responses might interact is more limited.

HIF-1 is a transcription factor and is critical to cellular survival in hypoxic conditions, both in cancer and cardiac cells. HIF-1 is composed of the O₂ and growth factor-regulated subunit HIF-1α, and the constitutively expressed HIF-1β subunit (arylhydrocarbon receptor nuclear translocator, ARNT), both of which belong to the basic helix-loop-helix (bHLH)-PAS (PER, ARNT, SIM) protein family In the human genome, three isoforms of the subunit of the transcription factor HIF have been identified: HIF-1, HIF-2 (also referred to as EPAS-1, MOP2, HLF, and HRF), and HIF-3 (of which HIF-32 also referred to as IPAS, inhibitory PAS domain).

Under normoxic conditions, HIF-1α is targeted for ubiquitinylation by pVHL and is rapidly degraded by the proteasome. This is triggered through post-translational HIF-1α hydroxylation on specific proline residues (proline 402 and 564 in human HIF-1α protein) within the oxygen dependent degradation domain (ODDD), by specific HIF-prolyl hydroxylases (HPH1-3-also referred to as PHD1-3) in the presence of iron, oxygen, and 2-oxoglutarate. The hydroxylated protein is then recognized by pVHL, which functions as an E3 ubiquitin ligase. The interaction between HIF-1α and pVHL is further accelerated by acetylation of lysine residue 532 through an N-acetyltransferase (ARD1). Concurrently, hydroxylation of the asparagine residue 803 within the C-TAD also occurs by an asparaginyl hydroxylase (also referred to as FIH-1), which by its turn does not allow the coactivator p300/CBP to bind to HIF-1 subunit In hypoxic conditions, HIF-1α remains not hydroxylated and does not interact with pVHL and CBP/p300.

Following hypoxic stabilization, HIF-1α translocates to the nucleus where it heterodimerizes with HIF-1β. The resulting activated HIF-1 drives the transcription of over 60 genes important for adaptation and survival under hypoxia including glycolytic enzymes, glucose transporters Glut-1 and Glut-3, endothelin-1 (ET-1), VEGF (vascular endothelial growth factor), tyrosine hydroxylase, transferrin, and erythropoietin (Brahimi-Horn et al., Trends Cell Biol. 11:S32-S36, 2001; Beasley et al., Cancer Res. 62:2493-2497, 2002; Fukuda et al., J. Biol. Chem. 277: 38205-38211, 2002; and Maxwell and Ratcliffe, Semin. Cell Dev. Biol. 13:29-37, 2002).

Quadri et al., in J. Med. Chem. 40:1561-1564, described antihypertensive steroids in which the C17 substituent is a furan ring (see also, U.S. Pat. Nos. 5,342,169; 5,489,582; 5,556,846; 5,567,694; 5,567,697; 5,591,734; and 5,593,982).

While HIF-1 is now understood to be the principal mediator of local, or cellular, responses to hypoxia, no global regulator of hypoxia has yet been recognized. It is an object of the invention to identify regulators of hypoxia, and further, to provide uses for such regulators.

### SUMMARY OF THE INVENTION

The present invention is based on the discovery that physiological and cellular responses to hypoxic stress are regulated, at least in part, by steroid signaling. At the cellular level, the pathway inhibits the normal hypoxic response which cells undergo to recruit blood vessels (e g inhibition of HIF-1 activation, VEGF secretion and/or angiogenesis), thereby separating systematic hypoxic response from local hypoxic response. At the level of the whole organism, signaling by steroids causes physiological changes, such as reduction of heart rate and increased blood pressure. While the role of steroids as regulators of responses to hypoxia has not been previously appreciated, many of the changes affected by such molecules appear to be orchestrated in a manner that favors the survival of major organs during periods of hypoxia. For example, blood flow is redirected away from the extremities to critical organs.

The present invention features a method of treating or preventing an ocular disorder in a mammal mediated by hypoxia inducible factor-1 (HIF-1) that includes administering to the mammal a steroid that modulates the effects of local and systemic hypoxic events for the treatment of ocular disorders. Dysregulation (e.g. excessive or insufficient signaling) of the HIF-steroid signaling pathway could be involved in the etiology of, or contribute in a downstream fashion to, ocular disorders, such as, angiogenic ocular disease, ocular inflammation, retinopathy, retinopathy of prematurity, macular degeneration, age related macular degeneration, contact lens overwear, corneal graft rejection, corneal neovascularization, choroidal neovascularization, corneal graft neovascularization, retinal neovascularization, cortical visual impairment, epidemic keratocon junctivitis, marginal keratolysis, Mooren ulcer, myopia, pars planitis, phylectenulosis, post-laser surgery complications, pterygium, radial keratotomy, retrolental fibroplasias, ocular ischemic syndrome, retinal ischemia, ischemic optic neuropathy, non-arteritic ischemic optic neuropathy, glaucoma, neovascular glaucoma, hypoxia related ocular surface inflammation, ocular or macular edema, ocular neovascular disease, superior limbic keratitis, Steven Johnson disease, Terrien's marginal degeneration, scleritis, radial keratotomy, uveitis, vitritis, myopia, optic pits, chronic retinal detachment, post-laser treatment complications, cataracts, cataract surgery, conjunctivitis, Stargardt's disease, Eale's disease, central retinal vein occlusion, sickle cell retinopathy, diabetic retinopathy, or any ocular disorder associated with hypotension, diabetes, angiogenic disorders, cancer (e.g., cancers of the eye), autoimmune disease (e.g., Behcet's disease), inflammatory conditions, atherosclerosis, stenosis of the carotid artery, Vitamin A deficiency, Stargardts disease, Wegeners sarcoidosis, or age-related metabolic changes.

In preferred embodiments, the steroid is a compound having the formula:

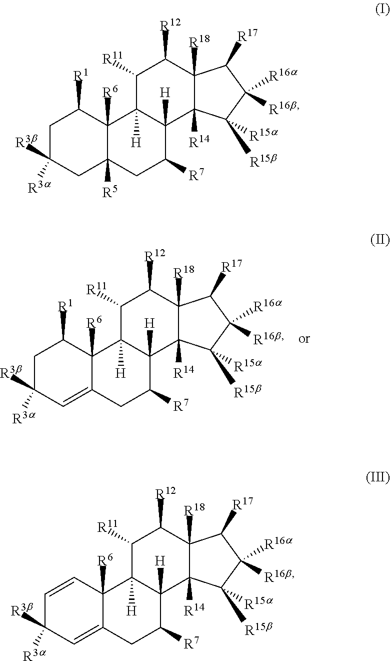

or a pharmaceutically acceptable salt or prodrug thereof, where

each of R¹, R⁵, R⁷, R₁₁, and R¹² is,independently, H; OH, OR^{1A}, or OC(O)R^{1A}, where R^{1A} is a substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl;

each of R^{3α} and R^{3β} is, independently, H, OH, OR^{3A}, OC(O)R^{3A}, or O-Sac, where each of R^{3A} and R^{3A} is, independently, a substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl, and Sac is a monosaccharide or a 1-4-linked di-, tri-, or tetrasaccharide unit comprising, in any order, one or more, monosaccharide units selected from the group consisting of: L-rhamnose, D-glucose, D-digitoxose, D-digitalose, D-digginose, D-sarmentose, L-vallarose, and D-fructose, where the linkage between any saccharide and the group attached to it can be by an α- or β-linkage, or R^{3α} and R^{3β} together are ═O, ═NNR^{3C}(CH₂)ₙNR^{3D}R^{3E}, or ═NO(CH₂)ₙNR^{3D}R^{3E}, where n is 2 to 6 and each of R^{3C}, R^{3D} and R^{3E} is, independently, H, a substituted or unsubstituted C₁₋₆ alkyl, substituted or unsubstituted C₁₋₄ alkaryl, or substituted or unsubstituted C₆₋₁₀ aryl, and with the proviso that at least one of R^{3α} and R^{3β} is not H;

R⁶ is CH₃, CH₂OR^{6A}, or CH₂OCOR^{6A}, where R^{6A} is H, substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl;

R¹⁴ is OH, Cl, OR^{14A}, or OC(O)R^{14A}, where R^{14A} is a substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl, or R¹⁴, R^{15β}, and the carbons they are bonded to together represent an epoxide;

each of R^{15α} and R^{15β} is, independently, H, OH, OR^{15A}, or OC(O)R^{15A}, where R^{15A} is a substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl, or R^{15α} and R¹⁵ together are ═O;

each of R^{16α} and R^{16β} is, independently, H, OH, OR^{16A}, or OC(O)R^{16A}, where R^{16A} is a substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl, or R^{16α} and R¹⁶ together are ═O;

R¹⁷ is

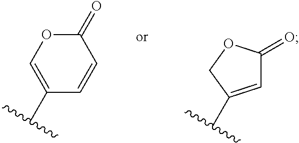

R¹⁸ is CH₃, CH₂OR^{18A}, or CH₂OCOR^{18A}, where R^{18A} is H, substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl;

providing that no carbon atom that is bonded to OH is bonded to another group via an oxygen bond and that said metabolic disorder is not diabetes.

In one embodiment, R^{3α} and R^{3β} together are ═NNR^{3C}(CH₂)ₙNR^{3D}R^{3E} or ═NO(CH₂)ₙNR^{3D}R^{3E}, where n is 2 to 6 and each of R^{3C}, R^{3D} and R^{3E} is, independently, H, a substituted or unsubstituted C₁₋₆ alkyl, substituted or unsubstituted C₁₋₄ alkaryl, or substituted or unsubstituted C₆₋₁₀ aryl.

Steroids that are useful as steroidal HIF-1 modulators include bufalin, 3α-hydroxybufalin, bufalin 3-acetate, bufalin 3-succinate, bufalin 3-methacrylate, bufalin 3-suberate, bufalin 3-methylsuberate, bufalin 3 [N-(tert-butoxycarbonyl)hydrazido]succinate, 3-oxobufalin, 14α-hydroxybufalin 3β,16β-diacetate, scillarenin, 3-oxoscillarenin, bufotalin, desacetylbufotalin, gamabufotalin, gamabufotalin 3-acetate, 3-oxogamabufotalin 11-acetate, telocinobufagin, hellebrigenin, acetylarenobufagin, 15α-hydroxybufalin, 15 α-hydroxybufalin 3-acetate, 15-oxobufalin 3-acetate, resibufagin, resibufaginol, resibufagenin, 3α-hydroxyresibufogenin, resibufagenin 3-acetate, 3-oxoresibufogenin, Δ¹-3-oxoresibufogenin, Δ^{1,4}-3-oxoresibufogenin, 16α-hydroxyresibufagenin 3-acetate, 14α,15α-epoxyresibufogenin, 3α-hydroxy-14α,15α-epoxyresibufogenin 3-acetate, 3-oxo-14α,15α-epoxyresibufogenin 3-acetate, 14α,15α-epoxyresibufogenin 3-acetate, 14α,15α-epoxyresibufogenin 3α-acetate, marinobufagin, periplogenin, digitoxigenin, digitoxigenin 3-acetate, digitoxigenin 3-suberate, digitoxigenin, 3-methylsuberate, Δ^{1,4}-digitoxigenin, cinobufagin, 3α-hydroxycinobufagin, cinobufagin 3-acetate, cinobufagin 3-succinate, cinobufagin 3-suberate, cinobufagin 3-cinnamate, 3-oxocinobufagin, cinobufagin 3,5-dinitrobenzoate, 3,16-diketocinobufagin, 16-oxocinobufagin 3-acetate, desacetylcinobufagin, desacetylcinobufagin 3-acetate, desacetylcinobufagin 3-acetate 16-succinate, desacetyl-14α,15α-cinobufagin 3-acetate, cinobufotalin, desacetylcinobufotalin, β-chlorohydrin, 14β-artebufogenin, 14β-artebufogenin 3-acetate, 14α-artebufogenin, 3-oxo-14α-artebufogenin, Δ^{1,4}-bufalin, Δ^{1,4}-3-oxobufalin, Δ^{1,4}-bufotalin 3-acetate, 7β-hydroxybufalin, 1β,7β-dihydroxybufalin, 16α-hydroxybufalin, 7β,16α-dihydroxybufalin, 3-epi-desacetylcinobufagin, 1β-hydroxy desacetylcinobufagin, 3-epi-desacetylcinobufotalin, cinobufagin 3-O-β-D-glucoside, 3-epi-7β-hydroxybufalin, telocinobufagin, 11β-hydroxybufalin, 15α-hydroxybufalin, 15β-hydroxybufalin, 12β-hydroxybufalin, 1β,12β-dihydroxybufalin, 12β-hydroxycinobufagin, 12β-hydroxy desacetylcinobufagin, 3-oxo-12β-hydroxycinobufagin, 3-oxo-12β-hydroxy desacetylcinobufagin, 12-oxo-cinobufagin, 3-oxo-12α-hydroxycinobufagin.

In certain other embodiments, steroidal HIF-1 modulators include digitoxigenin, digoxin, lanatoside C, Strophantin K, uzarigenin, desacetyllanatoside A, actyl digitoxin, desacetyllanatoside C, strophanthoside, scillaren A, proscillaridin A, digitoxose, gitoxin, strophanthidiol, oleandrin, acovenoside A, strophanthidine digilanobioside, strophanthidin-d-cymaroside, digitoxigenin-L-rhamnoside, digitoxigenin theretoside, strophanthidin, digoxigenin 3,12-diacetate, gitoxigenin, gitoxigenin 3-acetate, gitoxigenin 3,16-diacetate, 16-acetyl gitoxigenin, acetyl strophanthidin, ouabagenin, 3-epigoxigenin, neriifolin, acetylneriifolin cerberin, theventin, somalin, odoroside, honghelin, desacetyl digilanide, calotropin, calotoxin, convallatoxin, oleandrigenin, bufalin, periplocyrnarin, digoxin (CP 4072), strophanthidin oxime, strophanthidin semicarbazone, strophanthidinic acid lactone acetate, ernicyrnarin, sannentoside D, sarverogenin, sarmentoside A, and sarmentogenin.

In certain other embodiments, the steroidal HIF-1 modulator is ouabain or proscillaridin.

In another aspect, the invention features a method of treating or preventing an ocular disorder in a mammal mediated by hypoxia inducible factor-1 (HIF-1) that includes administering to the mammal a compound having the formula:

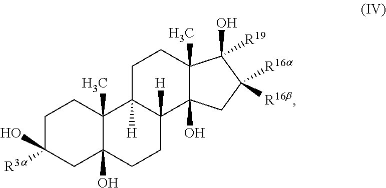

or a pharmaceutically acceptable salt or prodrug thereof, where

R^{3α} is H, CF₃, a substituted or unsubstituted C₁₋₆ alkyl, a substituted or unsubstituted C₂₋ alkenyl, or a substituted or unsubstituted C₂₋₆alkynyl;

each of R^{16α} and R^{16β} is, independently, H, OH, OR^{16A}, or OC(O)R^{16A}, where R^{16A} is a substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl, or R^{16a} and R¹⁶ together are ═O; and

R¹⁹ is

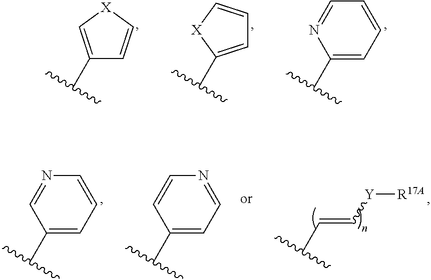

where X is O, S, NR^{17B}, S(O), or S(O)₂, and Y is O, NR^{17C}, C(O), C(O)O, OC(O), S(O)₂, or S(O)₂O, where each of R^{17A}, R^{17B}, and R^{17C} is, independently, H, a substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl;

providing that no carbon atom that is bonded to OH is bonded to another group via an oxygen bond.

Examples of compounds of formula IV include a compound selected from the group consisting of:

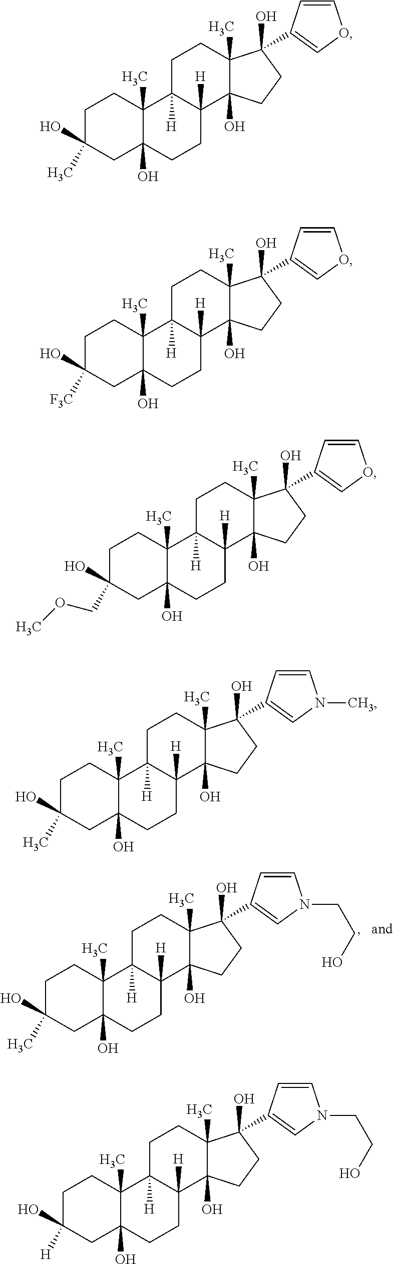

In another aspect, the invention features a method of treating or preventing an ocular disorder in a mammal mediated by HIF-1 that includes administering an effective amount of an agent to the mammal that antagonizes one or more elements of a pathway that leads to the endogenous biosynthesis of a cardiolide or bufadienolide, such as, for example, ouabain or proscillaridin. In preferred embodiments, the ocular disorder is characterized by ischemia.

Examples of ocular disorders associated with HIF-1 mediated local or systemic hypoxia include but are not limited to angiogenic ocular disease, ocular inflammation, retinopathy, retinopathy of prematurity, macular degeneration, age related macular degeneration, contact lens overwear, corneal graft rejection, corneal neovascularization, choroidal neovascularization, corneal graft neovascularization, retinal neovascularization, cortical visual impairment, epidemic keratocon junctivitis, marginal keratolysis, Mooren ulcer, myopia, pars planitis, phylectenulosis, post-laser surgery complications, pterygium, radial keratotomy, retrolental fibroplasias, ocular ischemic syndrome, retinal ischemia, ischemic optic neuropathy, non-arteritic ischemic optic neuropathy, glaucoma, neovascular glaucoma, hypoxia related ocular surface inflammation, ocular or macular edema, ocular neovascular disease, superior limbic keratitis, Steven Johnson disease, Terrien's marginal degeneration, scleritis, radial keratotomy, uveitis, vitritis, myopia, optic pits, chronic retinal detachment, post-laser treatment complications, cataracts, cataract surgery, conjunctivitis, Stargardt's disease, Eale's disease, central retinal vein occlusion, sickle cell retinopathy, diabetic retinopathy. In additional embodiments, the ocular disorder is associated with systemic hypoxia resulting from or causing hypotension, diabetes, angiogenic disorders, cancer (e.g., cancers of the eye), autoimmune disease (e.g., Behcet's disease), inflammatory conditions, atherosclerosis, stenosis of the carotid artery, Vitamin A deficiency, Stargardts disease, Wegeners sarcoidosis, or age-related metabolic changes. The ocular disorder can also be a disorder characterized by ischemia. Non-limiting examples of ocular disorders characterized by ischemia include ocular ischemic syndrome, retinal ischemia, ischemic optic neuropathy, non-arteritic ischemic optic neuropathy, glaucoma, and neovascular glaucoma.

The steroidal HIF-1 modulator compound of the invention can be administered by any means but is desirably formulated for ocular administration, for example by injection, topical application, or using an intraocular device. In preferred embodiments, the compound is formulated for sustained or controlled release of the compound. The steroidal HIF-1 modulator compound of the invention can also be administered in combination with anti-VEGF therapeutics such as VEGF antibodies (Genentech), and VEGF antagonists (see for example van Wijngaarden et al. JAMA 293:1509-1513 (2005)). Preferred anti-VEGF therapeutics include Macugen™ (Pfizer) and Lucentis™ (Genentech), which can be used as recommended by the manufacturer.

### DEFINITIONS

As used herein, the terms “alkyl” and the prefix “alk-” are inclusive of both straight chain and branched chain saturated or unsaturated groups, and of cyclic groups, i.e., cycloalkyl and cycloalkenyl groups. When an alkyl group is a saturated hydrocarbon it is, unless otherwise specified, from 1 to 6 carbons and is exemplified by methyl, ethyl, n- and iso-propyl, n-, sec-, iso- and tert-butyl, neopentyl. When an alkyl group is unsaturated it is, unless otherwise specified, from 2 to 12 carbons, such as, for example, 2 to 6 carbon atoms or 2 to 4 carbon atoms, containing one or more carbon-carbon double or triple bonds and is exemplified by ethenyl, 1-propenyl, 2-propenyl, 2-methyl-1-propenyl, 1-butenyl, 2-butenyl, ethynyl, 1-propynyl, and the like. When an alkyl group is cyclic it is, unless otherwise specified, from three to eight carbons and is exemplified by cyclopropyl, cyclobutyl, cyclopentyl, cyclohexyl, cycloheptyl, bicyclo[2.2.1]heptyl, and the like. Alkyl groups may be optionally substituted with one, two, three or, in the case of alkyl groups of two carbons or more, four substituents independently selected from the group consisting of (1) alkoxy of one to six carbon atoms; (2) alkylsulfinyl of one to six carbon atoms; (3) alkylsulfonyl of one to six carbon atoms; (4) alkynyl of two to six carbon atoms; (5) amino; (6) aryl; (7) arylalkoxy, where the alkylene group is of one to six carbon atoms; (8) azido; (9) cycloalkyl of three to eight carbon atoms; (10) halo; (11) heterocyclyl; (12) (heterocycle)oxy; (13) (heterocycle)oyl; (14) hydroxyl; (15) hydroxyalkyl of one to 6 carbons; (16) N-protected amino; (17) nitro; (18) oxo or thiooxo; (19) perfluoroalkyl of 1 to 4 carbons; (20) perfluoroalkoxyl of 1 to 4 carbons; (21) spiroalkyl of three to eight carbon atoms; (22) thioalkoxy of one to six carbon atoms; (23) thiol; (24) OC(O)R^{A}, where R^{A} is selected from the group consisting of (a) substituted or unsubstituted C₁₋₆ alkyl, (b) substituted or unsubstituted C₆ or C₁₀ aryl, (c) substituted or unsubstituted C₇₋₁₆ arylalkyl, where the alkylene group is of one to six carbon atoms, (d) substituted or unsubstituted C₁₋₉ heterocyclyl, and (e) substituted or unsubstituted C₂₋₁₅ heterocyclylalkyl, where the alkylene group is of one to six carbon atoms; (25) C(O)R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylalkyl, where the alkylene group is of one to six carbon atoms; (26) CO₂R^{B}, where R^{B} is selected from the group consisting of (a) hydrogen, (b) substituted or unsubstituted C₁₋₆ alkyl, (c) substituted or unsubstituted C₆ or C₁₀ aryl, (d) substituted or unsubstituted C₇₋₁₆ arylalkyl, where the alkylene group is of one to six carbon atoms, (e) substituted or unsubstituted C₁₋₉ heterocyclyl, and (f) substituted or unsubstituted C₂₋₁₅ heterocyclylallcyl, where the alkylene group is of one to six carbon atoms; (27) C(O)NR^{C}R^{D}, where each of R^{C} and R^{D} is, independently, selected from the group consisting of (a) hydrogen, (b) alkyl, (c) aryl and (d) arylalkyl, where the alkylene group is of one to six carbon atoms; (28) S(O)R^{E}, where R^{E} is selected from the group consisting of (a) alkyl, (b) aryl, (c) arylalkyl, where the alkylene group is of one to six carbon atoms, and hydroxyl; (29) S(O)₂R^{E}, where R^{E} is selected from the group consisting of (a) alkyl, (b) aryl, (c) arylalkyl, where the alkylene group is of one to six carbon atoms, and hydroxyl; (30) S(O)₂NR^{F}R^{G}, where each of R^{F} and R^{G} is, independently, selected from the group consisting of (a) hydrogen, (b) alkyl, (c) aryl and (d) arylalkyl, where the alkylene group is of one to six carbon atoms; and (31) —NR^{H}R^{I}, where each of R^{H} and R^{I} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms, (i) alkcycloalkyl, where the cycloalkyl group is of three to eight carbon atoms, and the alkylene group is of one to ten carbon atoms, (j) alkanoyl of one to six carbon atoms, (k) aryloyl of 6 to 10 carbon atoms, (l) alkylsulfonyl of one to six carbon atoms, and (m) arylsulfonyl of 6 to 10 carbons atoms, with the proviso that no two groups are bound to the nitrogen atom through a carbonyl group or a sulfonyl group.

By “C_{x-y} alkaryl” is meant a chemical substituent of formula —RR′, where R is an alkyl group of x to y carbons and R′ is an aryl group as defined elsewhere herein.

By “C_{x-y} alkheteraryl” is meant a chemical substituent of formula RR″, where R is an alkyl group of x to y carbons and R″ is a heteroaryl group as defined elsewhere herein.

The term “aryl,” as used herein, represents a mono- or bicyclic carbocyclic ring system having one or two aromatic rings and is exemplified by phenyl, naphthyl, 1,2-dihydronaphthyl, 1,2,3,4-tetrahydronaphthyl, fluorenyl, indanyl, indenyl and the like and may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) arylalkyl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) alkcycloalkyl, where the cycloalkyl group is of three to eight carbon atoms and the alkylene group is of one to ten carbon atoms; (22) halo; (23) haloalkyl of one to six carbon atoms; (24) heterocyclyl; (25) (heterocyclyl)oxy; (26) (heterocyclyl)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) (CH₂)_{q}CO₂R^{A}, where q is an integer of from zero to four and R^{A} is selected from the group consisting of (a) alkyl, (b) aryl and (c) arylalkyl, where the alkylene group is of one to six carbon atoms; (37) (CH₂)_{q}C(O)NR^{B}R^{C}, where R^{B} and R^{C} are independently selected from the group consisting of (a) hydrogen, (b) alkyl, (c) aryl and (d) arylalkyl, where the alkylene group is of one to six carbon atoms; (38) (CH₂)_{q}S(O)₂R^{D}, where R^{D} is selected from the group consisting of (a) alkyl, (b) aryl and (c) arylalkyl, where the alkylene group is of one to six carbon atoms; (39) (CH₂)_{q}S(O)₂NR^{E}R^{F}, where each of R^{E} and R^{F} is, independently, selected from the group consisting of (a) hydrogen, (b) alkyl, (c) aryl and (d) arylalkyl, where the alkylene group is of one to six carbon atoms; (40) (CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

By “bufadienolide” is meant any compound having a steroid backbone, a hydroxy group at the C3 position of the steroidal A ring, and a six-membered doubly unsaturated lactone ring substituent at C17 of the steroidal D-ring. Examples of bufadienolides are compounds of formulas I, II, or III, as described herein, where R¹⁷ is:

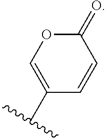

The term “carbonyl” as used herein, represents a C(O) group, which can also be represented as C═O.

By “cardiolide” is meant any compound having a steroid backbone, a hydroxy group at the C3 position of the steroidal A ring, and a five-membered unsaturated lactone ring substituent at C17 of the steroidal D-ring. Examples of cardiolides are those compounds of formulas I, II, or III, as described herein, where R¹⁷ is:

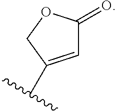

By “effective amount” is meant the amount of a compound required to treat or prevent a disorder mediated by a local or general hypoxic response. The effective amount of active compound(s) used to practice the present invention for therapeutic or prophylactic treatment of conditions caused by or contributed to by a hypoxic response varies depending upon the manner of administration, the age, body weight, and general health of the subject. Ultimately, the attending physician or veterinarian will decide the appropriate amount and dosage regimen. Such amount is referred to as an “effective” amount.

The term “halogen” or “halo,” as used interchangeably herein, represents F, Cl, Br and I.

The term “heteroaryl,” as used herein, represents that subset of heterocycles, as defined herein, which are aromatic: i.e., they contain 4n+2 pi electrons within the mono- or multicyclic ring system. Exemplary unsubstituted heteroaryl groups are of from 1 to 9 carbons.

The terms “heterocycle” or “heterocyclyl,” as used interchangeably herein represent a 5-, 6- or 7-membered ring, unless otherwise specified, containing one, two, three, or four heteroatoms independently selected from the group consisting of nitrogen, oxygen and sulfur. The 5-membered ring has zero to two double bonds and the 6- and 7-membered rings have zero to three double bonds. The term “heterocycle” also includes bicyclic, tricyclic and tetracyclic groups in which any of the above heterocyclic rings is fused to one or two rings independently selected from the group consisting of an aryl ring, a cyclohexane ring, a cyclohexene ring, a cyclopentane ring, a cyclopentene ring and another monocyclic heterocyclic ring such as indolyl, quinolyl, isoquinolyl, tetrahydroquinolyl, benzofuryl, benzothienyl and the like. Heterocyclics include pyrrolyl, pyrrolinyl, pyrrolidinyl, pyrazolyl, pyrazolinyl, pyrazolidinyl, imidazolyl, imidazolinyl, imidazolidinyl, pyridyl, piperidinyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, uricyl, thiadiazolyl, pyrimidyl, tetrahydrofuranyl, dihydrofuranyl, tetrahydrothienyl, dihydrothienyl, dihydroinidolyl, tetrahydroquinolyl, tetrahydroisoquinolyl, pyranyl, dihydropyranyl, dithiazolyl, benzofuranyl, benzothienyl and the like. Heterocyclic groups also include compounds of the formula

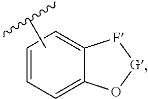

where

F′ is selected from the group consisting of CH₂, CH₂O and O, and G′ is selected from the group consisting of C(O) and (C(R′)(R″))ᵥ, where each of R′ and R″ is, independently, selected from the group consisting of hydrogen or alkyl of one to four carbon atoms, and v is one to three and includes groups such as 1,3-benzodioxolyl, 1,4-benzodioxanyl and the like. Any of the heterocycle groups mentioned herein may be optionally substituted with one, two, three, four or five substituents independently selected from the group consisting of: (1) alkanoyl of one to six carbon atoms; (2) alkyl of one to six carbon atoms; (3) alkoxy of one to six carbon atoms; (4) alkoxyalkcyl, where the alkyl and alkylene groups are independently of one to six carbon atoms; (5) alkylsulfinyl of one to six carbon atoms; (6) alkylsulfinylalkyl, where the alkyl and alkylene groups are independently of one to six carbon atoms; (7) alkylsulfonyl of one to six carbon atoms; (8) alkylsulfonylalkyl, where the alkyl and alkylene groups are independently of one to six carbon atoms; (9) aryl; (10) arylalkyl, where the alkyl group is of one to six carbon atoms; (11) amino; (12) aminoalkyl of one to six carbon atoms; (13) aryl; (14) arylalkyl, where the alkylene group is of one to six carbon atoms; (15) aryloyl; (16) azido; (17) azidoalkyl of one to six carbon atoms; (18) carboxaldehyde; (19) (carboxaldehyde)alkyl, where the alkylene group is of one to six carbon atoms; (20) cycloalkyl of three to eight carbon atoms; (21) alkcycloalkyl, where the cycloalkyl group is of three to eight carbon atoms and the alkylene group is of one to ten carbon atoms; (22) halo; (23) haloalkyl of one to six carbon atoms; (24) heterocycle; (25) (heterocycle)oxy; (26) (heterocycle)oyl; (27) hydroxy; (28) hydroxyalkyl of one to six carbon atoms; (29) nitro; (30) nitroalkyl of one to six carbon atoms; (31) N-protected amino; (32) N-protected aminoalkyl, where the alkylene group is of one to six carbon atoms; (33) oxo; (34) thioalkoxy of one to six carbon atoms; (35) thioalkoxyalkyl, where the alkyl and alkylene groups are independently of one to six carbon atoms; (36) (CH₂)_{q}CO₂R^{A}, where q is an integer of from zero to four and R^{A} is selected from the group consisting of (a) alkyl, (b) aryl and (c) arylalkyl, where the alkylene group is of one to six carbon atoms; (37) (CH₂)_{q}C(O)NR^{B}R^{C}, where each of R^{B} and R^{C} is, independently, selected from the group consisting of (a) hydrogen, (b) alkyl, (c) aryl and (d) arylalkyl, where the alkylene group is of one to six carbon atoms; (38) (CH₂)_{q}S(O)₂R^{D}, where R^{D} is selected from the group consisting of (a) alkyl, (b) aryl and (c) arylalkyl, where the alkylene group is of one to six carbon atoms; (39) (CH₂)_{q}S(O)₂NR^{E}R^{F}, where each of R^{E} and R^{F} is, independently, selected from the group consisting of (a) hydrogen, (b) alkyl, (c) aryl and (d) arylalkyl, where the alkylene group is of one to six carbon atoms; (40) (CH₂)_{q}NR^{G}R^{H}, where each of R^{G} and R^{H} is, independently, selected from the group consisting of (a) hydrogen; (b) an N-protecting group; (c) alkyl of one to six carbon atoms; (d) alkenyl of two to six carbon atoms; (e) alkynyl of two to six carbon atoms; (f) aryl; (g) arylalkyl, where the alkylene group is of one to six carbon atoms; (h) cycloalkyl of three to eight carbon atoms and (i) alkcycloalkyl, where the cycloalkyl group is of three to eight carbon atoms, and the alkylene group is of one to ten carbon atoms, with the proviso that no two groups are bound to the nitrogen atom through a carbonyl group or a sulfonyl group; (41) oxo; (42) thiol; (43) perfluoroalkyl; (44) perfluoroalkoxy; (45) aryloxy; (46) cycloalkoxy; (47) cycloalkylalkoxy; and (48) arylalkoxy.

The term “hydroxy” or “hydroxyl,” as used interchangeably herein, represents an —OH group.

The term “hydroxyalkyl,” as used herein, represents an alkyl group, as defined herein, substituted by one to three hydroxy groups, with the proviso that no more than one hydroxy group may be attached to a single carbon atom of the alkyl group and is exemplified by hydroxymethyl, dihydroxypropyl and the like.

By “hypoxia” is generally meant a shortage of oxygen. By “ischemia” is meant a shortage in the blood supply to organ. For both hypoxia and ischemia, the shortage can be absolute or relative to the amount required by the recipient organ or tissue. Ischemia can result in hypoxia when the shortage in the blood supply results in a shortage in oxygen.

By “ocular disorder” is meant any disease or disorder of the eye, including the sclera, iris, cornea, pupil, lens, conjuctiva, vitreous, choroids, optic nerve, macular, and retina, associated with local or systemic hypoxia. Non-limiting examples of ocular disorders include angiogenic ocular disease, ocular inflammation, retinopathy, retinopathy of prematurity, macular degeneration, age related macular degeneration, contact lens overwear, corneal graft rejection, corneal neovascularization, choroidal neovascularization, corneal graft neovascularization, retinal neovascularization, cortical visual impairment, epidemic keratocon junctivitis, marginal keratolysis, Mooren ulcer, myopia, pars planitis, phylectenulosis, post-laser surgery complications, pterygium, radial keratotomy, retrolental fibroplasias, ocular ischemic syndrome, retinal ischemia, ischemic optic neuropathy, non-arteritic ischemic optic neuropathy, glaucoma, neovascular glaucoma, hypoxia related ocular surface inflammation, ocular or macular edema, ocular neovascular disease, superior limbic keratitis, Steven Johnson disease, Terrien's marginal degeneration, scleritis, radial keratotomy, uveitis, vitritis, myopia, optic pits, chronic retinal detachment, post-laser treatment complications, cataracts, cataract surgery, conjunctivitis, Stargardt's disease, Eale's disease, central retinal vein occlusion, sickle cell retinopathy, diabetic retinopathy, or any ocular disorder associated with hypotension, diabetes, angiogenic disorders, cancer (e.g., cancers of the eye), autoimmune disease (e.g., Behcet's disease), inflammatory conditions, atherosclerosis, stenosis of the carotid artery, Vitamin A deficiency, Stargardts disease, Wegeners sarcoidosis, or age-related metabolic changes.

The term “pharmaceutically acceptable salt,” as used herein, represents those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, S. M Berge et al. describe pharmaceutically acceptable salts in detail in J. Pharmaceutical Sciences 66:1-19, 1977. The salts can be prepared in situ during the final isolation and purification of the compounds of the invention or separately by reacting the free base group with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like. As used herein, the terms “to prevent,” “preventing,” or “prevention” refer to any reduction, no matter how slight, of a subject's predisposition or risk for a condition mediated by the presence or absence of hypoxia inducible factor-1.

The term “prodrug,” as used herein, represents compounds that are rapidly transformed in vivo to a parent compound of the above formula, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996), each of which is incorporated herein by reference. The term “pharmaceutically acceptable prodrugs” as used herein, represents those prodrugs of the compounds of the present invention which are, within the scope of sound medical judgement, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

As used herein, the term “steroidal HIF-1 modulator” means those compounds that include a steroid core with either a pyrone or butenolide substituent at C17 (the “pyrone form” and “butenolide form”). Additionally, steroidal HIF-1 modulators may optionally be glycosylated at C3. For example, steroidal HIF-1 modulators and include one to four sugars attached to the 3β-OH group. The sugars most commonly used include L-rhamnose, D-glucose, D-digitoxose, D-digitalose, D-digginose, D-sarmentose, L-vallarose, and D-fructose. In general, the sugars affect the pharmacokinetics of a steroidal HIF-1 inhibitor with little other effect on biological activity. For this reason, aglycone forms of steroidal HIF-1 modulators are available and are intended to be encompassed by the term “steroidal HIF-1 modulator,” as used herein. The pharmacokinetics of a steroidal HIF-1 modulator may be adjusted by adjusting the hydrophobicity of the molecule, with increasing hydrophobicity tending to result in greater absorption and an increased half-life. Sugar moieties may be modified with one or more groups, such as, for example, an acetyl group.

As used herein, the terms “treating,” “treatment,” “treated,” or “to treat” mean to alleviate symptoms, eliminate the causation either on a temporary or permanent basis, or to alter or slow the appearance of symptoms or symptom worsening. The term “treatment” includes alleviation or elimination of causation of a condition mediated by the presence or absence of hypoxia inducible factor-1.

### BRIEF DESCRIPTION OF THE DRAWINGS

FIG. 1 is a western blot showing the anti-hypoxia properties of ouabain (BNC1) and proscillaridin (BNC4) in ocular disease. BNC1 and BNC4 inhibit hypoxia-mediated HIF-1α induction in a human retinal pigment epithelium cell line (ARPE-19).

FIG. 2A is an angiogenesis antibody membrane array showing the expression of VEGF, angiogenenin and TIMP-1 after treatment with BNC4 and incubation under hypoxic conditions. FIG. 2B is a graph showing the effect of BNC4 on the expression of VEGF under normoxic and hypoxic conditions in ARPE-19 cells. FIG. 2C is a graph showing the IC₅₀ for BNC4 on VEGF expression under hypoxic conditions in ARPE-19 cells.

FIG. 3A is a graph showing the effect of BNC4 on the expression of TIMP-1 under normoxic and hypoxic conditions in ARPE-19 cells. FIG. 3B is a graph showing the IC₅₀ for BNC4 on TIMP-1 expression under hypoxic conditions in ARPE-19 cells. FIG. 3C is a graph showing the effect of BNC4 on the expression of angiogenin under normoxic and hypoxic conditions in ARPE-19 cells. FIG. 3D is a graph showing the IC₅₀ for BNC4 on angiogenin expression under hypoxic conditions in ARPE-19 cells.

FIGS. 4A-4C are a series of images showing the effects of BNC-1, BNC4, and vehicle control in a choroidal neovascularization model using an Alzet osmotic pump. FIG. 4D is a graph showing the area of choroidal neovascularization in eyes treated with BNC1, BNC4, and vehicle control. The serum concentration of BNC 1 was 20 ng/ml and the serum concentration of BNC4 was 60 ng/ml.

### DETAILED DESCRIPTION

The present invention is based in part on the discovery that the administration of certain agents, such as, for example, ouabain (BNC1) or proscillaridin (BNC4), to mammalian subjects retard the suite of effects that are observed as a result of cellular or systemic hypoxia. Therefore, such compounds may be used in a tailored manner to modulate one or more of such effects in a clinical setting. For example, the steroids of formula I, formula II or formula III, as described herein, can modulate hypoxia-mediated cellular or systemic activities, including those mediated by HIF-1, and therefore be used in the prevention or treatment of ocular disorders, particularly ocular disorders associated with systemic or local hypoxic stress.

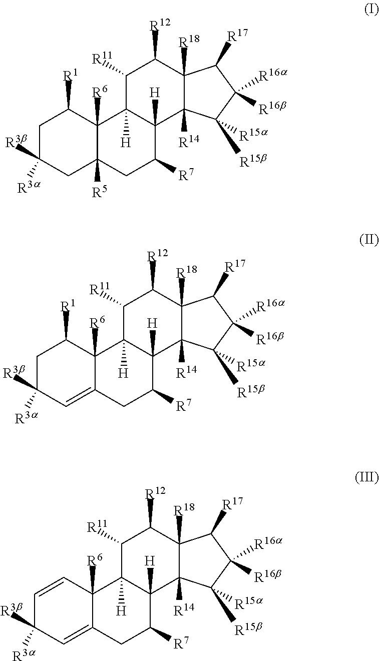

Many bufadienolide or cardiolide steroids have been previously described, such as, for example, those described by Kamano et al., in J. Med. Chem. 45:5440-5447, 2002; Kamano et al., in J. Nat. Prod. 65:1001-1005, 2002; Nogawa et al., in J. Nat. Prod. 64:1148-1152, 2001; and Qu et al., J. Steroid Biochem. Mol. Biol. 91:87-98.

In addition, several different routes to the preparation of bufadienolides have been described in the art, including Soncheimer et al., J. Am. Chem. Soc. 91:1228-1230, 1969; Stache et al., Tetrahedron Lett. 35:3033-3038, 1969; Pettit et al., Can. J. Chem. 47:2511, 1969; Pettit et al., J. Org. Chem. 35:1367-9, 1970; Tsay et al., Heterocycles 12:1397-1402, 1979; Sen et al., J. Chem. Soc. Chem. Comm. 66:1213-1214, 1982; Wiesner et al., Helv. Chim. Acta 66:2632-2641, 1983; Weisner & Tsai, Pure and Appl. Chem. 53:799-810, 1986, U.S. Pat. Nos. 4,001,402; 4,102,884; 4,175,078; 4,242,332; and 4,380,624.

The present invention also features steroids that bind to the Na+/K+ ATPase receptor to inhibit this enzyme, and as a result modulate the effects of local and systemic hypoxic events.

The invention also features a method of treating or preventing an ocular disorder in a mammal associated with local or systemic hypoxia that includes administering to the mammal a compound having the formula:

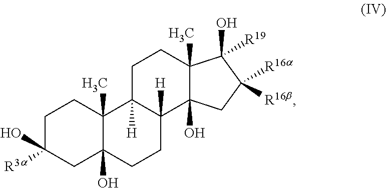

where the definitions of R^{3α}, R^{16α}, R^{16β} and R¹⁹ are provided elsewhere herein.

### Inhibition of Cardiolide or Bufadienolide Biosynthesis

The depletion of oxygen supply due to obstructed or inadequate blood supply is the common pathological state associated with various ocular tissue ischemias, including but not limited to, ocular ischemic syndrome, retinal ischemia, ischemic optic neuropathy, non-arteritic ischemic optic neuropathy, glaucoma, neovascular glaucoma. The alleviation of tissue ischemia is critically dependent upon angiogenesis, the process by which new capillaries are generated from existing vasculature and tissue. The spontaneous growth of new blood vessels provide collateral circulation in and around an ischemic area, improves blood flow, and alleviates the symptoms caused by the ischemia. Although surgery or angioplasty may help to revascularize ischemic regions in some cases, the extent, complexity and location of the arterial lesions which cause the occlusion often prohibits such treatment

In one embodiment, the present invention features a method of treating or preventing an ocular disorder in a mammal characterized by ischemia and mediated by hypoxia inducible factor-1 (HIF-1). The method involves administering an agent to the mammal that antagonizes one or more elements, in particular enzymes, of a pathway that leads to the endogenous biosynthesis of a cardiolide or bufadienolide. Examples include those ocular disorders that are treated or prevented by the expression of a cellular proliferation factor (e.g., cyclin G2, IGF-2, IGF-BP1, IGF-BP2, IGF-BP3, EGF, WAF-1, TGF-α, or TGF-β2); a cell survival factor (e.g., ADM, IGF2, IGF-BP1, IGF-BP2, IGF-BP3, NOS2, TGF-α, or VEGF); an angiogenesis factor (EG-VEGF, ENG, LEP, LRP1, TGF-β3, or VEGF); a glucose metabolism factor (HK1, BK2, AMF/GP1, ENO1, GLUT1, GAPDH, LDHA, PFKBF3, or PRKL); a cell adhesion factor (e.g., MIC1); or an apoptosis factor (e.g., NIP3, NIX, or RTP801), where the expression of the factor is increased after the agent is administered to the mammal.

Accordingly, cardiolide or bufadienolide biosynthesis pathway inhibition can be affected to treat a variety of ocular disorders characterized by ischemia including but not limited to ocular ischemic syndrome, retinal ischemia, ischemic optic neuropathy, non-arteritic ischemic optic neuropathy, glaucoma, and neovascular glaucoma.

### Ocular Disorders Mediated by the Hypoxic Response

The steroidal HIF-1 modulators of the invention are useful for the treatment of ocular disorders such as those associated with systemic hypoxic response disorders. Non-limiting examples of such disorders include hypotension, diabetes, angiogenic disorders, cancer (e.g., cancers of the eye), autoimmune disease (e.g., Behcet's disease), inflammatory conditions, atherosclerosis, stenosis of the carotid artery, Vitamin A deficiency, Stargardts disease, Wegeners sarcoidosis, or age-related metabolic changes. The ocular disorder can also be a disorder characterized by ischemia. Non-limiting examples of an ocular disorder characterized by ischemia include ocular ischemic syndrome, retinal ischemia, ischemic optic neuropathy, non-arteritic ischemic optic neuropathy, glaucoma, and neovascular glaucoma.

Detailed examples of the use of the steroidal HIF-1 modulators described herein for the treatment of particular ocular diseases are described below and are intended to illustrate the invention but not to limit the invention. It should be noted that many ocular disorders can be included in more than one category described below, for example, tumors of the eye can be included as ocular disorders associated with angiogenesis and proliferative diseases.

Proliferative Diseases

The inventors have demonstrated that the steroidal HIF-1 modulators described herein are effective in suppressing hypoxia-induced gene expression, such as VEGF expression in cancer cells. Examples of cancers of the eye include primary intraocular cancers such as melanoma, primary intracellular lymphoma, retinoblastoma, medulloepithelioma, neovascular glaucoma, and secondary intraocular cancers that have spread to the eye from another part of the body.

For example, steroidal HIF-1 modulators are effective in suppressing VEGF, EGF, insulin and/or IGF-responsive gene expression in various growth factor responsive cancer cell lines. As another example, the inventors have observed that steroidal HIF-1 modulators are effective in suppressing HIF-responsive gene expression in cancer cell lines and furthermore, these compounds are shown to have potent antiangiogenesis effects in certain cell lines.

Notably, steroidal HIF-1 modulators can affect proliferation of cancer cell lines at a concentration well below the known toxicity level. The IC₅₀ measured for ouabain across several different cancer cell lines ranged from about 15 nM to about 600 nM, or about 80 nM to about 300 nM. The concentration at which a steroidal HIF-1 modulator is effective as part of an antiproliferative treatment may be further decreased by combination with an additional agent that negatively regulates HIF-responsive genes, such as a redox effector or a steroid signal modulator. For example, as shown herein, the concentration at which a HIF-1 inhibitor (e.g., ouabain or proscillaridin) is effective for inhibiting proliferation of cancer cells is decreased 5-fold by combination with a steroid signal modulator (Casodex). Therefore, in certain embodiments, the invention provides combination therapies of HIF-1 inhibitor with, for example, steroid signal modulators and/or redox effectors. Additionally, HIF-1 inhibitors may be combined with radiation therapy, taking advantage of the radiosensitizing effect of many HIF-1 inhibitors.

Proliferative Retinopathies

In one aspect of the invention, a steroidal HIF-1 modulator, as described herein, may be administered to retinal tissue for the treatment of proliferative retinopathies. It is known that VEGF causes retinal neovascularization in animals including human beings suffering from diabetic retinopathy and steroidal HIF-1 modulators may act by down-regulating HIF-1 activity and/or VEGF expression. Diabetic retinopathy is a common microvascular complication in patients with type 1 diabetes. The progression of background retinopathy to proliferative retinopathy leads to visual impairment through bleeding or retinal detachment by accompanying fibrous tissues. The invention provides a method to treat diabetic retinopathy or other proliferative retinopathies in a patient that includes administering to a retina of the patient a composition containing a steroidal HIF-1 modulator, as described herein, at an amount/level sufficient to down-regulate VEGF expression in the retina and inhibit angiogenesis in the retina.

Experiments in animal models with induced ocular neovascularization show that VEGF is up-regulated several fold before the formation of new blood vessels, and that blocking its action inhibits retinal neovascularization. Also, increased vascular permeability is a characteristic sign of early stages (background retinopathy) of diabetic retinopathy, and VEGF is up-regulated during this stage. Retinal digest preparations from diabetic animals and humans show scattered capillary occlusions which is a stimulus for increased vascular permeability. VEGF is such a vascular permeability factor.

A diabetic rat model of experimental retinopathy may be used to screen candidate HIF-1 modulators, and to test and/or verify the efficacy of a candidate HIF-1 modulators in the retinal tissue. Such a diabetic rat model of retinopathy is known to one skilled in the art. For example, chronic hyperglycemia can be induced in 4-6 week old Wistar rats by intravenous injection of 60-65 mg/kg body weight streptozotocin. Diabetes can be monitored consecutively by taking body weight and blood glucose levels into consideration.

To illustrate, when these rats reach, for example, a body weight of about 330 g and their blood glucose levels of 25 mmol/l, the subject steroidal HIF-1 modulator, as described herein, can be administered to the retinal tissue at 1 to 2 week intervals. The age-matched nondiabetic rats are used as controls. VEGF levels can be monitored in the retinal tissues of diabetic and control rats at regular intervals of 7 to 14 days, by any of the suitable techniques such as in situ hybridization for VEGF, immunoreactivity, immunohistochemistry and western blot analysis. For example, retinal protein extracts can be performed to confirm the relative decrease in VEGF protein levels in retinal tissue. The treatments are continued until VEGF levels in the retinal extracts are similar to that in nondiabetic rats. Quantitation of cellular capillaries can also be performed in diabetic rats and compared to that of the controls. Thus, therapies that include the use of a steroidal HIF-1 modulator provide an effective anti-VEGF strategy in diabetic retinopathy. Therapies that include the use of a steroidal HIF-1 modulator can also be used in combination with anti-VEGF compounds such as anti-VEGF antibodies or VEGF antagonists.

Angiogenesis

As noted elsewhere herein, the present invention describes steroids that are potent inhibitors of HIF-1, which is itself a potent activator of pro-angiogenic factors. While not wishing to be bound to any particular mechanism, it is reasonable to expect that a factor involved in mounting a global response to hypoxia would suppress local responses, such as angiogenesis, that would be inappropriate if local cellular hypoxia is attributable to systemic disturbances in ventilation or oxygen supply. It is intriguing to note that endogenous steroids are produced by the avascular tissues of the eye lens, and that removal of cataract tissue is often associated with undesirable vascularization of the lens. The discoveries provided herein suggest that the endogenous steroids in the lens play a direct role in suppressing vascularization of the eye, and may therefore be useful in treating various proliferative retinopathies.

The present methods can be used to inhibit angiogenesis which is nonpathogenic; i.e., angiogenesis which results from normal biological processes in the subject. The present methods can also inhibit angiogenesis which is associated with an angiogenic disease; i.e., a disease in which pathogenicity is associated with inappropriate or uncontrolled angiogenesis. For example, most cancerous solid tumors generate an adequate blood supply for themselves by inducing angiogenesis in and around the tumor site. This tumor-induced angiogenesis is often required for tumor growth, and also allows metastatic cells to enter the bloodstream.

Angiogenic diseases associated with ocular disorders include retinal neovascularization, choroidal neovascularization, diabetic retinopathy, retinopathy of prematurity (ROP), macular degeneration, age-related macular degeneration (ARMD), atherosclerosis, cancers, and inflammatory diseases. Most, if not all of these diseases are characterized by the destruction of normal tissue by newly formed blood vessels in the area of (diseased) neovascularization. For example, in ARMD, the choroid is invaded and destroyed by capillaries. The angiogenesis-driven destruction of the choroid in ARMD eventually leads to partial or full blindness.

In one example, the invention provides a method to treat choroidal neovascularization in a patient. This method involves delivering to subretinal space or retinal pigment epithelium of the patient a composition containing a steroidal HIF-1 modulator, as described herein, in an amount sufficient to down-regulate VEGF expression in said tissue and inhibit angiogenesis in the choroidal tissue.

A salient feature of the present invention is the discovery that certain agents induce a hypoxic stress response and expression of angiogenic factors (such as VEGF) in cells, and that a steroidal HIF-1 modulator, as described herein, can be used to reduce that response. Since hypoxic stress response is associated with the expression of certain angiogenesis factors, including (but not limited to) VEGF, inhibiting hypoxic stress response would also inhibit VEGF- (and other angiogenesis factor-) mediated angiogenesis.

Choroidal Neovascularization

In another aspect, the methods, reagents, and pharmaceutical compositions of the present invention can be used to inhibit choroidal neovascularization (CNV). CNV is a serious complication of age related macular degeneration and it is characterized by the growth of new blood vessels from the choroid, through the Buch's membrane into the subretinal space. This ultimately leads to the formation of choroidal neovascular membranes from which blood and serum may leak, causing vision loss. At present, age-related macular degeneration is clinically difficult to treat.

It is known that VEGF is a causative agent in a variety of ocular angiogenic diseases including age-related macular degeneration. For example, it has been shown that the overexpression of VEGF in retinal pigment epithelial cells is sufficient to induce CNV (Spilsbiry et al. Am J Pathol 1257:135-144, 2000).

The animal models of choroidal neovascularization in the subretinal space are well known in the art (Tobe et al. J. Jpn. Ophthalmol. Soc 98:837-845, 1994; Shen et al., Br. J. Ophthamomol. 82:1062-1071, 1998). For example, a rat with CNV can be administered with a subject steroidal HI F-1 modulator, as described herein, with or without other anti-angiogenesis therapeutic agents. Such a treatment protocol may be used to determine whether it is sufficient to down-regulate VEGF expression and inhibit CNV in the rat.

Briefly, the CNV rats can be used for subretinal administration of the subject steroidal HIF-1 modulator (with or without other therapeutic agents). The animals are anesthetized, for example, by a mixture of ketamine and xylazine administered intramuscularly. The eyes can be further treated with topical amethocaine drops and the pupils dilated with 1% tropicamide and 2.5% phenylephrine hydrochloride drops. The conjunctiva can be cut close to the limbus to expose the sclera. A 32 gauge needle is then passed through this hole in a tangential direction under an operating microscope, to deliver the agents to the subretinal space Immediately after the subretinal injection a circular bleb is usually observed under the operating microscope. The success of each subretinal injection is further confirmed by the observation of a partial retina detachment as seen by indirect ophthalmoscopy. The needle is kept in the subretinal space for 1 minute, withdrawn gently, and antibiotic ointment applied to the wound site.

VEGF levels can be determined by VEGF mRNA expression in RPE cells. In addition, to determine whether administering an agent in the RPE has down-regulated VEGF, which VEGF expression would otherwise have a vasopermeabilty effect on blood vessels, fluorescein angiograms can be used to detect vascular leakage. Fluorescein angiography in the context of CNV is well known in the art. For example, fluorescein angiograms 5-10 days post-subretinal injection of the agent(s) can be performed to determine areas of vascular leakage.

Thus the subject steroidal HIF-1 modulators, as described herein, provides an ideal system for targeted anti-angiogenic gene therapy in the eye.

Cataract Surgery

In normal lenses, immunoreactivity against bufalin and ouabain-like factor is sevenfold to 30-fold higher in the capsular epithelial layer than in the lens fiber region (Lichtstein et al., Involvement of Na⁺,K⁺-ATPase inhibitors in cataract formation, in Na/K-ATPase and Related ATPases, 2000, Taniguchi, K. & Haya, S., eds, Elsevier Science, Amsterdam). In human cataractous lenses, the concentration of the sodium pump inhibitor was much higher than in normal lenses. Hence, it was isolated from cataractous lenses and identified as 19-norbufalin and its Thr-Gly-Ala tripeptide derivative (Lichtstein et al., Eur. J. Biochem. 216:261-268, 1993). The steroids described herein alter the osmotic balance of lenses and induce cataract formation by crystalline degradation and protein leakage that initiate opacity. On the other hand, cataract surgery will remove such steroids, thus may also lose the local inhibitory effect to undesirable angiogenesis in the eye. Patients after cataract surgery may therefore be more vulnerable to conditions associated with abnormal angiogenesis. The subject compounds (or in combination with other anti-angiogenesis factors) may help to prevent or alleviate the risk or symptoms of such situations.

### Administration of Steroidal HIF-1 Modulators

The present invention also features pharmaceutical compositions of a steroidal HIF-1 modulator, as described herein, and a pharmaceutically acceptable excipient. Compositions containing at least one compound of the invention that is suitable for use in human or veterinary medicine may be presented in forms permitting administration by a suitable route. These compositions may be prepared according to the customary methods, using one or more pharmaceutically acceptable adjuvants or excipients. The adjuvants comprise, inter alia, diluents, sterile aqueous media, and various non-toxic organic solvents. Acceptable carriers or diluents for therapeutic use (e.g., saline) are well known in the pharmaceutical field, and are described, for example, in Remington: The Science and Practice of Pharmacy (20th ed.), ed. A. R. Gennaro, Lippincott Williams & Wilkins, 2000, Philadelphia, ASHP Handbook on Injectable Drugs, 11^{th} edition, ed. Trissel, ASHP, Maryland, 2001, and Encyclopedia of Pharmaceutical Technology, eds. J. Swarbrick and J. C. Boylan, 1988-1999, Marcel Dekker, New York. The compositions may be presented in the form of tablets, pills, granules, powders, aqueous solutions or suspensions, injectable solutions, elixirs, or syrups, and the compositions may optionally contain one or more agents chosen from the group comprising sweeteners, flavorings, colorings, and stabilizers in order to obtain pharmaceutically acceptable preparations.

It is not intended that the administration of a steroidal HIF-1 modulator of the invention to a mammal, including humans, be limited to a particular mode of administration, dosage, or frequency of dosing. The present invention contemplates all modes of administration including, but not limited to ocular, oral, parenteral by intravenous injection, transdermal, inhalation, implantation, or intramuscular injection.

Desirably, the steroidal HIF-1 modulator is administered directly to the eye in any form suitable for ocular drug administration, e.g., as a solution or suspension for administration as eye drops, injection, or eye washes, as a topical formulation (e.g., an ointment), or in an ocular insert (e.g., intraocular device) that can be implanted in the conjunctiva, sclera, pars plana, anterior segment, or posterior segment of the eye. Implants can provide for sustained or controlled release of the formulation to the ocular surface, typically over an extended time period.

Topical formulations for ocular administration are well known to those of skill in the art. The use of patches, corneal shields (see, e.g., U.S. Pat. No. 5,185,152), and ophthalmic solutions (see, e.g., U.S. Pat. No. 5,710,182) and ointments, e.g., eye drops, is also within the skill in the art. If desired, the subject steroidal HIF-1 modulator can be administered non-invasively using a needleless injection device, such as the Biojector 2000 Needle-Free Injection Management System™ available from Bioject, Inc.

Alternatively, the steroidal HIF-1 modulator can be administered using, for example, intravitreal injection or subretinal injection, optionally preceded by a vitrectomy. Subretinal injections can be administered to different compartments of the eye (e.g., the anterior chamber or posterior chamber).

In some embodiments, it is advantageous to deliver the subject steroidal HIF-1 modulator via periocular (e.g., episcleral, sub-tenon, or sub-conjunctival) injection. For example, most standard injection techniques require puncturing layers of the eye, including the sclera, choroid, and retina. To minimize trauma to those layers of the eye, the steroidal HIF-1 modulator can be administered into the sub-tenon (i.e., episcleral) space surrounding the scleral portion of the eye. The sub-tenon space is enclosed by Tenon's capsule, a fibrous sheath encasing the posterior segment of the eye. Puncture of this fibrous sheath with an injection device is less traumatic to the layers of the eye responsible for vision. Due to the structure of Tenon's capsule, the exposure of non-ocular cells to the steroidal HIF-1 modulator is limited. Sub-tenon injection also allows the administration of a greater volume of therapeutic composition compared to that allowed by, for example, subretinal injection.

The steroidal HIF-1 modulator can be administered via an ophthalmologic instrument for delivery to a specific region of an eye, e.g., the sub-tenon space. The use of a specialized ophthalmologic instrument ensures precise administration of the steroidal HIF-1 modulator, while minimizing damage to adjacent ocular tissue. Delivery of the steroidal HIF-1 modulator to a specific region of the eye also limits exposure of unaffected cells to the steroidal HIF-1 modulator, thereby reducing the risk of side effects. A preferred ophthalmologic instrument is a combination of forceps and subretinal needle or sharp bent cannula.

In most cases, sub-tenon delivery of a composition to the eye involves surgically opening Tenon's capsule and injecting into the sub-tenon space using a syringe or cannula. Alternatively, Tenon's capsule is grasped by the practitioner, not surgically opened, and the therapeutic composition is injected into the sub-tenon space using, for example, a syringe. The steroidal HIF-1 modulator can be administered to other regions of the ocular apparatus such as, for instance, the ocular muscles, the orbital fascia, the eye lid, the lacrimal apparatus, and the like as is appropriate.

The steroidal HIF-1 modulator of the invention is preferably present in or on a device that allows controlled or sustained release, such as an ocular sponge, meshwork, mechanical reservoir, or mechanical implant. Implants (see, e.g., U.S. Pat. Nos. 4,853,224, 4,997,652, and 5,443,505), devices (see, e.g., U.S. Pat. Nos. 4,863,457, 5,098,443, 5,554,187, and 5,725,493), such as an implantable device, e.g., a mechanical reservoir, an intraocular device, or an extraocular device with an intraocular conduit, especially an implant or a device comprised of a polymeric composition, are particularly useful for ocular administration of the therapeutic factor or nucleic acid sequence encoding the therapeutic factor. Intraocular devices slowly release non-toxic therapeutic levels of various pharmaceutical agents. Such devices can be implanted anywhere in the eye, including the anterior chamber or vitreous cavity.

Examples of such controlled or sustained release devices and methods for delivering drugs to the eye are known in the art. Examples of various controlled-release devices which are biocompatible and can be implanted into the eye are described in U.S. Pat. No. 6,331,313. In certain embodiments, the devices described therein have a core comprising a drug and a polymeric outer layer which is substantially impermeable to the entrance of an environmental fluid and substantially impermeable to the release of the drug during a delivery period, and drug release is effected through an orifice in the outer layer. These devices have an orifice area of less than 10% and can be used to deliver a variety of drugs with varying degrees of solubility and or molecular weight. The rate of release of the drug is determined solely by the composition of the core and the total surface area of the one or more orifices relative to the total surface area of the device. In certain embodiments, the biocompatible, implantable ocular controlled-release drug delivery device is sized for implantation within an eye for continuously delivering a drug within the eye for a period of at least several weeks. Such device comprises a polymeric outer layer that is substantially impermeable to the drug and ocular fluids, and covers a core comprising a drug that dissolves in ocular fluids, wherein the outer layer has one or more orifices through which ocular fluids may pass to contact the core and dissolve drug, and the dissolved drug may pass to the exterior of the device.

Another type of ocular insert is an implant in the form of a monolithic polymer matrix that gradually releases the formulation to the eye through diffusion and/or matrix degradation. With such an insert, it is preferred that the polymer be completely soluble and or biodegradable (i.e., physically or enzymatically eroded in the eye) so that removal of the insert is unnecessary. These types of inserts are well known in the art, and are typically composed of a water-swellable, gel-forming polymer such as collagen, polyvinyl alcohol, or a cellulosic polymer.

Another type of insert that can be used to deliver the present formulation is a diffusional implant in which the formulation is contained in a central reservoir enclosed within a permeable polymer membrane that allows for gradual diffusion of the formulation out of the implant. Osmotic insert may also be used, i.e., implants in which the formulation is released as a result of an increase in osmotic pressure within the implant following application to the eye and subsequent absorption of lachrymal fluid. In one example, the steroidal HIF-1 modulator is administered to a patient using an osmotic pump, such as the Alzet® Model 2002 osmotic pump. Osmotic pumps provide continuous delivery of test agents, thereby eliminating the need for frequent, round-the-clock injections. With sizes small enough even for use in mice or young rats, these implantable pumps have proven invaluable in predictably sustaining compounds at therapeutic levels, avoiding potentially toxic or misleading side effects.

To meet different therapeutic needs, Alzet's osmotic pumps are available in a variety of sizes, pumping rates, and durations. At present, at least ten different pump models are available in three sizes (corresponding to reservoir volumes of 100 μL, 200 μL and 2 mL) with delivery rates between 0.25 μL/hr and 10 μL/hr and durations between one day to four weeks.

While the pumping rate of each commercial model is fixed at manufacture, the dose of agent delivered can be adjusted by varying the concentration of agent with which each pump is filled. Provided that the animal is of sufficient size, multiple pumps may be implanted simultaneously to achieve higher delivery rates than are attainable with a single pump. For more prolonged delivery, pumps may be serially implanted with no ill effects. Alternatively, larger pumps for larger patients, including human and other non-human mammals may be custom manufactured by scaling up the smaller models.

Additional examples of ocular implant methods and devices, and related improvements for drug delivery in the eye are described in U.S. Pat. Nos. 6,589,999, 6,579,519, 5,824,072, 5,776,445, 5,766,242, 5,632,984, 5, 443,505, and 5,902,598; U.S. Patent Application publications US20040175410A1, US20040151754A1, US20040237068, US20040022853A1, US20030203030A1, and PCT publications WO9513765A1, WO0130323A2, WO0202076A2, WO0243785A2, and WO04026106A2.

Examples of commercially available intraocular devices include the Vitrasert™ (Bausch & Lomb), which is an intravitreal implant currently used for the delivery of ganciclovir in patients with AIDS-related CMV retinitis. The Vitrasert™ implant contains gangciclovir embedded in a polymer-based system that slowly releases the drug. The steroidal HIF-1 modulator of the present invention can be embedded in the same such polymer-based system or any other acceptable carrier for slow release of the steroidal HIF-1 modulator. The implant, surgically placed in the posterior segment of the eye, allows diffusion of the drug locally to the site of infection over an extended period of months.

Another example of a commercially available intraocular device is Surodex™ (Oculex Pharmaceuticals), which is an intraocular implant currently used for delivery of dexamethasone. The Surodex™ device can also be used for the controlled delivery of the steroidal HIF-1 modulator of the invention.

For any of the intraocular devices described herein, implantation normally requires only local anesthesia and is conducted in an outpatient, day-surgery setting. The implant can be removed when depleted of drugs, for example, usually within five to eight months for Vitrasert™, and a new implant can be inserted.

The materials are formulated to suit the desired route of administration. The formulation may comprise suitable excipients include pharmaceutically acceptable buffers (e.g., saline), stabilizers, local anesthetics, and the like that are well known in the art. For example, the steroidal HIF-1 modulator formulation can be incorporated into a sterile ocular insert that provides for sustained or controlled release of the formulation over an extended time period, generally in the range of about 12 hours to 60 days, and possibly up to 12 months or more, following implantation of the insert into the conjunctiva, sclera, or pars plana, or into the anterior segment or posterior segment of the eye. Sustained release formulations are known in the art (see, e.g., U.S. Pat. No. 5,378,475) and comprise, for example, gelatin, chondroitin sulfate, a polyphosphoester, such as bis-2-hydroxyethyl-terep-hthalate (BHET), or a polylactic-glycolic acid. In one example, International Patent Application WO02087586A1 discloses a sustained release system that includes a polymer and a prodrug having a solubility less than about 1 mg/ml dispersed in the polymer. Advantageously, the polymer is permeable to the prodrug and may be non-release rate limiting with respect to the rate of release of the prodrug from the polymer. This permits improved drug delivery within a body in the vicinity of a surgery via sustained release rate kinetics over a prolonged period of time, while not requiring complicated manufacturing processes.

Dosage levels of active ingredients in the pharmaceutical compositions of the invention may be varied to obtain an amount of the active compound(s) that achieves the desired therapeutic response for a particular patient, composition, and mode of administration. The selected dosage level depends upon the activity of the particular compound, the route of administration, the severity of the condition being treated, and the condition and prior medical history of the patient being treated. For adults, the doses are generally from about 0.001 to about 100 mg/kg, desirably about 0.01 to about 50 mg/kg body weight per day, more desirably 0.01 to 10 mg/kg body weight per day, and most desirably 0.1 to 1 mg/kg body weight per day.

For ocular administration, the preferred serum concentration of the steroidal HIF-1 modulator dosage will generally range from 0.1 to 100 ng/ml, preferably about 1.0 to 100 ng/ml and most preferably about 10 to 50 ng/ml serum concentration. For example, BNC 1 is administered in a dosage that produces a serum concentration of about 20 ng/ml and BNC 4 is administered in a dosage that produces a serum concentration of about 15 ng/ml.

Multiple applications of the steroidal HIF-1 modulator can also be used when needed. For example, at least two applications of the steroidal HIF-1 modulator can be administered to the same eye. Preferably, the multiple doses are administered while retaining steroidal HIF-1 modulator concentrations above background levels. Also preferably, the ocular cell is contacted with two applications via direct administration to the eye within about 30 days or more. More preferably, two or more applications are administered to ocular cells of the same eye within about 90 days or more. However, three, four, five, six, or more doses can be administered in any time frame (e.g., 2, 7, 10, 14, 21, 28, 35, 42, 49, 56, 63, 70, 77, 85, or more days between doses) needed to treat or prevent the ocular disorder mediated by a local or general hypoxic response. Doses are determined for each particular case using standard methods in accordance with factors unique to the patient, including age, weight, general state of health, and other factors which can influence the efficacy of the compound(s) of the invention.

### EXMAPLES

The following examples are for illustrative purpose only, and should in no way be construed to be limiting in any respect of the claimed invention.

The exemplary HIF-1-modulating compounds used in following studies are referred to as BNC1 and BNC4.

BNC1 is ouabain or g-Strophanthin (STRODIVAL®), which has been used for treating myocardial infarction. It is a colorless crystal with predicted IC₅₀ of about 0.06-0.35 μg/mL and max. plasma concentration of about 0.03 μg/mL. According to the literature, its plasma half-life in human is about 20 hours, with a range of between 5-50 hours. Its common formulation is injectable. The typical dose for current indication (i.v.) is about 0.25 mg, up to 0.5 mg/day.

BNC4 is proscillaridin (TALUSIN®), which has been approved for treating chronic cardiac insufficiency in Europe. It is a colorless crystal with predicted IC₅₀ of about 0.01-0.06 μg/mL and max. plasma concentration of about 0.1 μg/mL. According to the literature, its plasma half-life in human is about 40 hours. Its common available formulation is a tablet of 0.25 or 0.5 mg. The typical dose for current indication (p.o.) is about 1.5 mg/day. In one embodiment, the dosage of BNC4 used in the methods of the application would result in 2×-4×, preferably 3× the IC₅₀ for secretion of VEGF (see Example 2).

The following materials and methods were used for the experiments described below.

Cells and Kits

The Eye cell line ARPE-19 was obtained from the American Type Culture Collection (ATCC, Manassas, Va.); Angiogenesis Antibody Array was purchased from Panomics, Inc. (Redwood City, Calif.); VEGF ELISA kit was purchased from PIERCE ENDOGEN (Rockford, Ill.); TM-1 and angiogenin (ANG) ELISA kits were purchased from R & D Systems (Minneapolis, Minn.).

Cell Culture

ARPE-19 was cultured in DMEM/F-12 medium supplemented with 10% heat-inactivated FBS, penicillin (100 U/mL), and streptomycin (100 ug/mL). Cells were grown in incubator with humidified atmosphere containing 5% CO₂ at 37° C. To induce hypoxia conditions, cells are placed in a Billups-Rothenberg modular incubator chamber and flushed with artificial atmosphere gas mixture (5% carbon dioxide, 1% oxygen, and balance nitrogen). The hypoxia chamber was then placed in a 37° C. incubator.

Western Blots

For HIF1-alpha Western blots, ARPE-19 cells were seeded in growth medium at a density of 7×10⁶ cells per 100 mm dish. Following 24-hour incubation, cells were subjected to hypoxic conditions for 4 hours to induce HIF1-alpha expression together with 1 uM BNC-1. The cells were harvested and lysed using the Mammalian Cell Lysis kit (Sigma, M-0253). The lysates were centrifuged to clear insoluble debris, and total protein contents were analyzed with BCA protein assay kit (Pierce, 23225). Samples were fractionated on 3-8% Tris-Acetate gel (Invitrogen NUPAGE system) by sodium dodecyl sulfate (SDS)-polyacrylamide gel electropherosis and transferred onto nitrocellulose membrane. HIF1-alpha protein was detected with anti-HIF1-alpha monoclonal antibody (BD Transduction Lab, 610959) at a 1:500 dilution with an overnight incubation at 4C in Tris-buffered solution-0.1% Tween 20 (TBST) containing 5% dry non-fat milk. Anti-Beta-actin monoclonal antibody (Abcam, ab6276-100) was used at a 1:5000 dilution with a 30-minute incubation at room temperature. Immunoreactive proteins were detected with stabilized goat-anti mouse HRP conjugated antibody (Pierce, 1858413) at a 1:10,000 dilution. The signal was developed using the West Femto substrate (Pierce, 34095).

Angiogenesis Antibody Array

APRE-19 cells were plated in four 10 cm² dishes at a confluence of 80% and cultured overnight. The next day, BNC4 was added into two of the four dishes at a final concentration of 40 nM. One dish without BNC4 and one with BNC4 were incubated in normal condition and the remaining two were incubated in hypoxia condition. After 24 hours incubation, supernatants were collected for the Angiogenesis Antibody Array. The Array experiment was carried out according to the manufacture's protocol. Briefly, four array membranes were placed one to one in 4 individual wells of the tray supplied and incubated in Blocking Buffer (3 mL for each membrane) for one hour at room temperature; membranes were rinsed twice with 1× Wash Buffer II (stock supplied) and incubated with four supernatants (2 mL each) accordingly for two hours; membranes were washed three times with Wash Buffer I and once with Wash Buffer II, 4 mL and 5 minutes per wash; membranes were incubated with diluted Biotin-Conjugated Angiogenesis Antibody Mix (prepared with supplied stocks) for one hour at room temperature. Membranes were washed as before and incubated with Strepavidin-HRP working solution (stock supplied) for one hour at room temperature; membranes were washed as before and incubated with Detection Buffer (stocks supplied) for 5 minutes; membranes were wrapped with plastic sheet and exposed to X-ray film or chemiluminescence imaging system.

ELISA

APRE-19 cells were plated in two 96-well plates at a confluence of 80% and cultured overnight. The next day, a series dilution of BNC4 (100, 50, 25, 12.5, 6.25, 3.13, 1.56, 0.78, 0 nM) was added into cultures. One plate was incubated in normal condition and the other plated was incubated in hypoxia condition for 24 hours. Supernatants were collected for ELISAs and cells were saved for MTS assay (MTS assay readouts was used in normalizing cell numbers). VEGF, TIMP-1 and angiogenin ELISAs were carried out according to the manufacture's protocols. Affection curves (BNC4 on secretion of angiogenesis proteins) and their IC₅₀s were generated by plotting the average normalized absorbance (450 nm minus 540nm) for each treatment on Y axis versus the corresponding BNC4 concentrations on X axis using software XLfit 4.1.

### Example 1

### Cardiac Glycoside Compounds Inhibits HIF-1α Expression

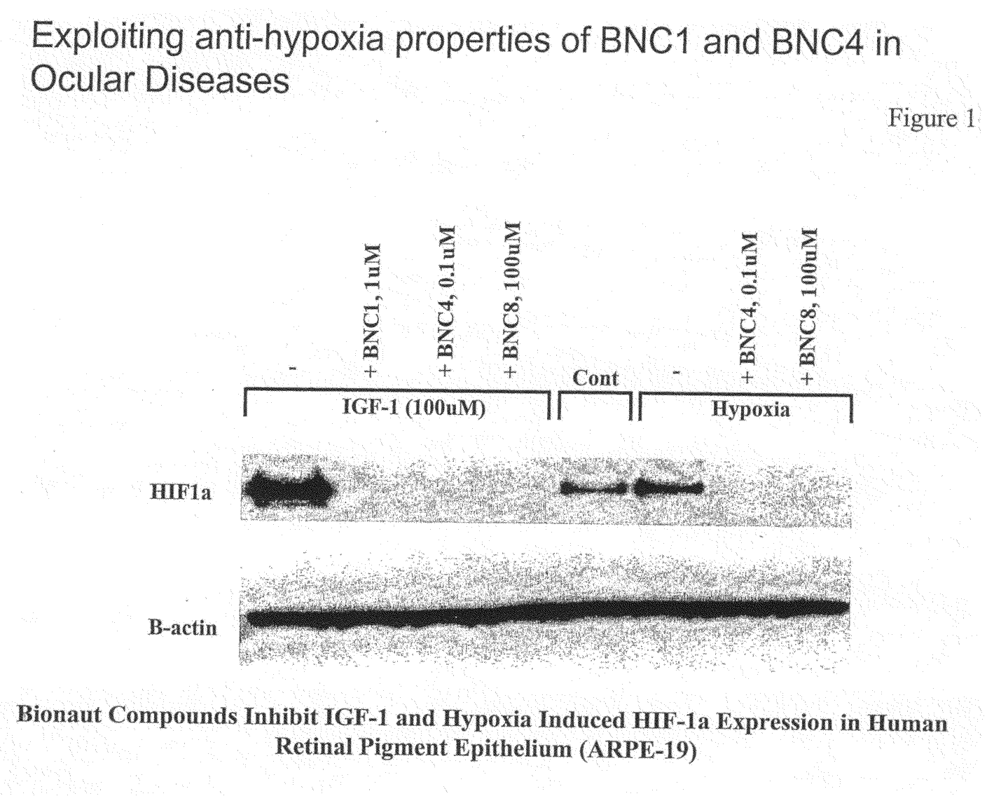

The ability of BNC1 and BNC4 to inhibit IGF-1 and hypoxia-induced HIF1α induction in human retinal pigment epithelium cells. FIG. 1 shows the result of immunoblotting for HIF-1α and β-actin (control) expression in ARPE-19 cells treated with BNC1 or BNC4 under hypoxia or after treatment with IGF-1. For these experiments, ARPE-19 cells were seeded in growth medium 24 hours prior to treatment. To show that BNC-1 inhibits HIF1-alpha expression in a concentration dependent manner, cells are treated with 1 uM (˜600 ng/ml) BNC-1 together with the indicated amount of MG132 under hypoxic conditions for 4 hours. To understand specifically the impact of BNC-1 on HIF-1 alpha synthesis, ARPE-19 cells were treated with MG132 and 1 uM BNC under normoxic conditions for the indicated time points. The observed expression is accounted by protein synthesis.

I examined the role of BNC-1 on the degradation rate of HIF-1 alpha. 24 hours prior to treatment, ARPE-19 cells were seeded in growth medium. The cells were placed in hypoxic conditions for four hours for HIF1-α accumulation. The protein synthesis inhibitor, cycloheximide (100 uM) together with 1 uM BNC-1 are added to the cells and kept in hypoxic conditions for the indicated time points.

To induce HIF1-alpha expression using an iron chelator, L-mimosine is added to ARPE-19 cells, seeded 24 hours prior, and placed under normoxic conditions for 24 hours

The results indicate that BNC4 is even more potent (about 10-times more potent) than BNC1 in inhibiting HIF-1α expression in human retinal pigment epithelial cells.

### Example 2

### BNC4 Inhibition of Angiogenic Factors in Human Retinal Pigment Epithelial Cell Line

In this study the effect of BNC4 on hypoxia induced expression of angiogenic factors was measured in human retinal pigment epithelial cell lines.

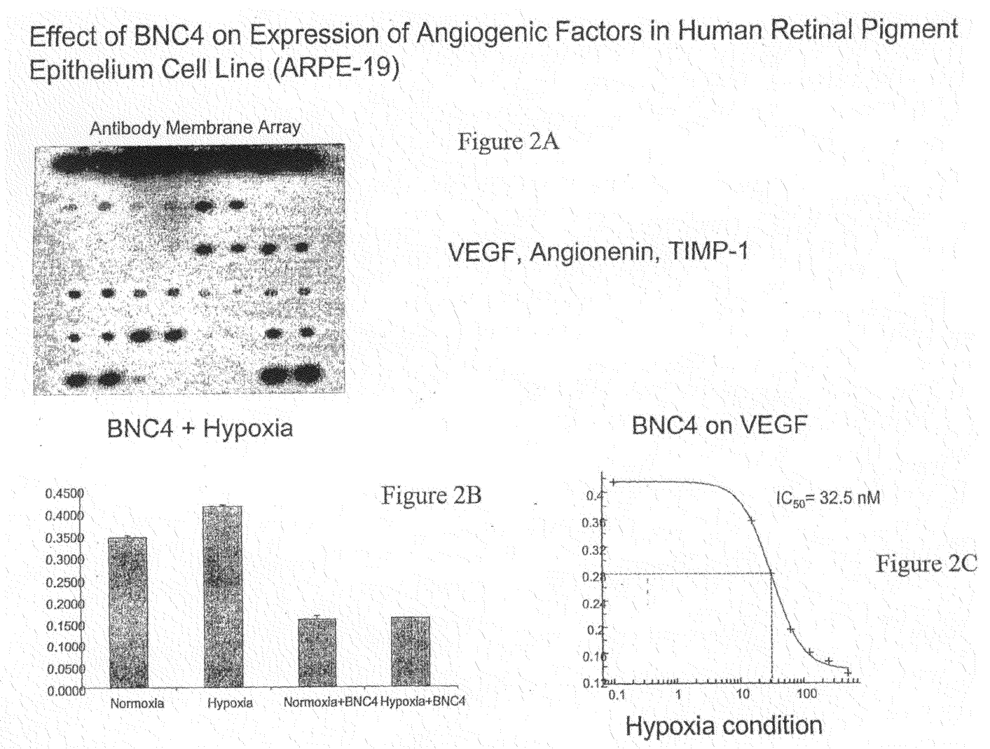

In the angiogenesis antibody membrane array experiment represented in FIG. 2A, ARPE-19 cells were grown to 80% confluence and then two of the four dishes were treated with BNC at a final concentration of 40 nM. One dish with BNC4 and one without for each condition was incubated under normal conditions and one of each dish was incubated under hypoxic conditions. The angiogenesis antibody array was carried out on the supernatants of each plate according to the manufacturer's instructions.

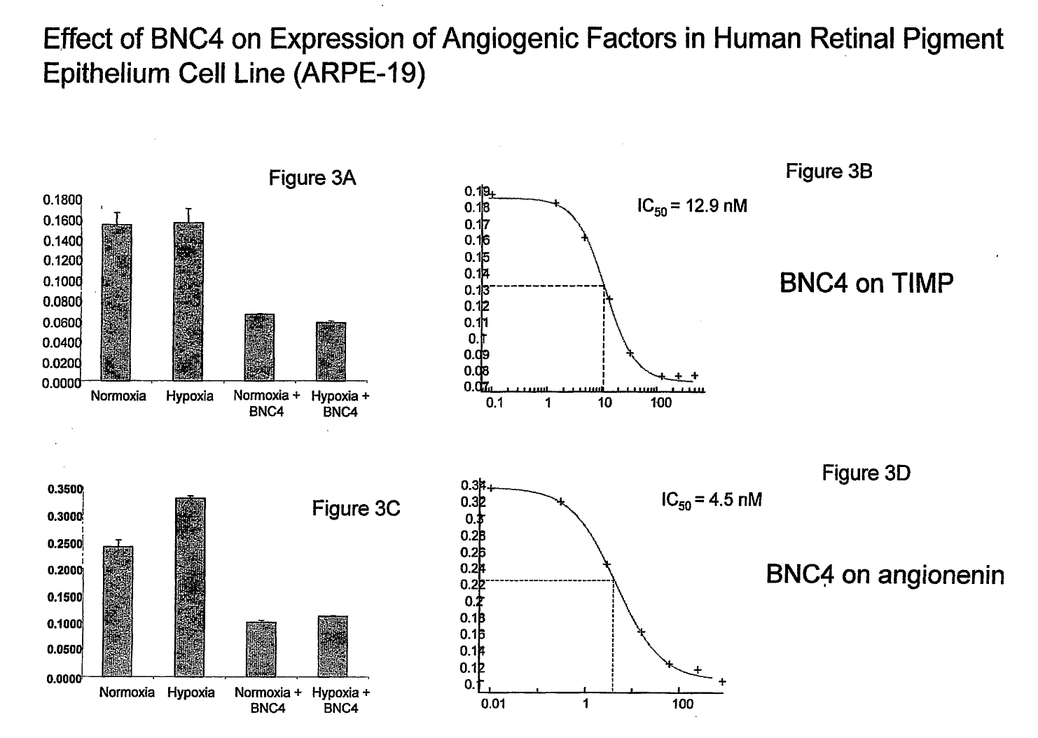

ELISAs were then performed on the APRE-19 cells treated with BNC4 and incubated under normal or hypoxia conditions as described above. For these experiments, APRE-19 cells were plated in two 96-well plates at a confluence of 80% and cultured overnight. The next day, a series dilution of BNC4 (100, 50, 25, 12.5, 6.25, 3.13, 1.56, 0.78, 0 nM) was added into cultures. One plate was incubated in normal condition and the other plate was incubated in hypoxia condition for 24 hours. Supernatants were collected for ELISAs and cells were saved for MTS assay (MTS assay readouts was used in normalizing cell numbers). VEGF (FIGS. 2B and 2C), TIMP-1 (FIGS. 3A and 3B), and angiogenin (FIGS. 3C and 3D). ELISAs were carried out according to the manufacture's protocols. Affection curves (BNC4 on secretion of angiogenesis proteins) and their IC₅₀s were generated by plotting the average normalized absorbance (450 nm minus 540 nm) for each treatment on Y axis vs the corresponding BNC4 concentrations on X axis using software Xlfit

The results of these experiments indicate that BNC4 inhibits the hypoxia-induced expression of angiogenic factors in a human retinal pigment epithelium cell line. The IC₅₀ for BNC4 for each of the factors were determined to be as follows: VEGF=32.5 nM, TIMP-1=12.9 nM, and angiogenin=4.5 nM.

### Example 3

### The effect of BNC1 and BNC4 in a choroidal neovascularization model.

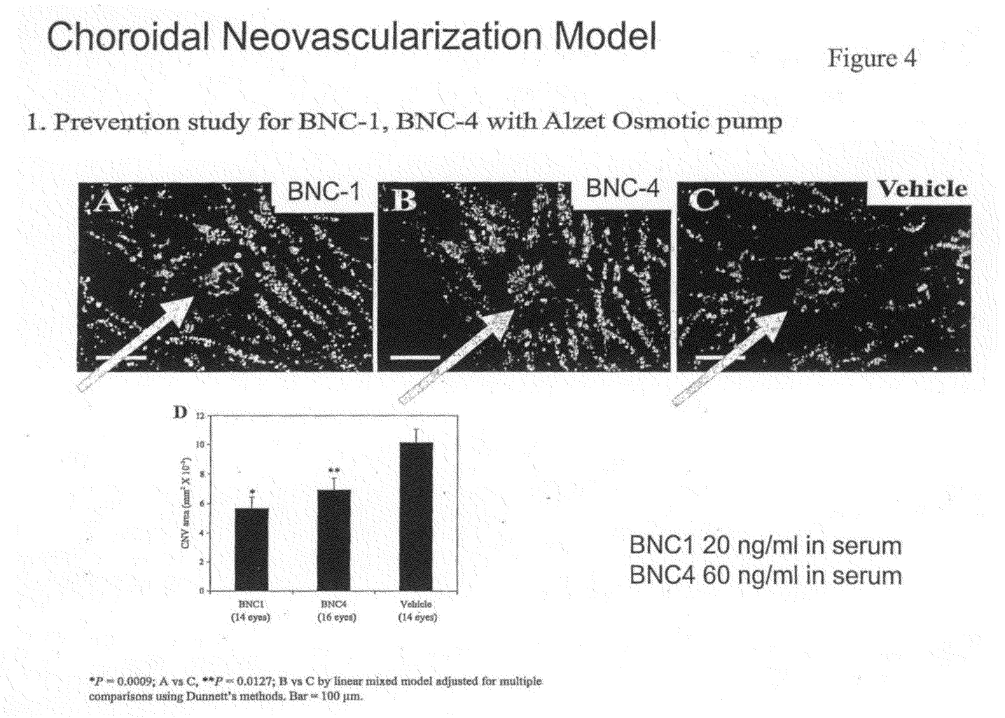

The preventive abilities of BNC1 and BNC4 were examined using a choroidal neovascularization model and an Alzet osmotic pump. The serum concentration of BNC1 in this experiment was 20 ng/ml and the serum concenration of BNC4 was 60 ng/ml. As shown in FIGS. 4A-4D, the use of BNC1 and BNC4, when administered using an Alzet osmotic pump reduced the area of choroidal neovascularization over vector alone in the model.

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims

What is claimed is:

1. A method of treating or preventing an ocular disorder in a mammal mediated by hypoxia inducible factor-1 (HIF-1), said method comprising administering to said mammal an effective amount of a compound having the formula: 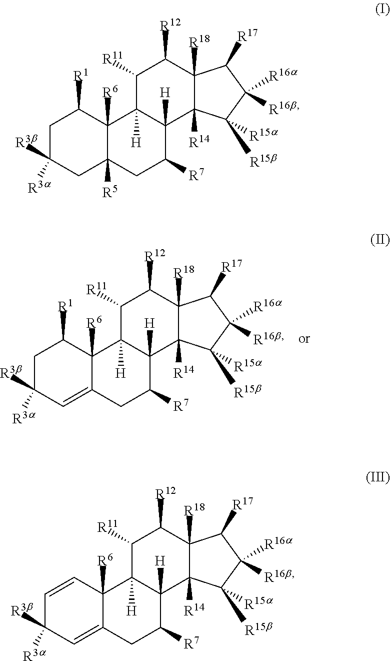
or a pharmaceutically acceptable salt or prodrug thereof, wherein
each of R¹, R⁵, R⁷, R¹¹, and R¹² is, independently, H; OH, OR^{1A}, or OC(O)R^{1A}, where R^{1A} is a substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl;
each of R^{3α} and R^{3β} is, independently, H, OH, OR^{3A}, OC(O)R^{3B}, or O-Sac, where each of R^{3A} and R^{3B} is, independently, a substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl, and Sac is a monosaccharide or a 1-4-linked di-, tri-, or tetrasaccharide unit comprising, in any order, one or more, monosaccharide units selected from the group consisting of: L-rhamnose, D-glucose, D-digitoxose, D-digitalose, D-digginose, D-sarmentose, L-vallarose, and D-fructose, wherein the linkage between any saccharide and the group attached to it can be by an α- or β-linkage, or R^{3α} and R^{3β} together are ═O, ═NNR^{3C}(CH₂)ₙNR^{3D}R^{3E}, or ═NO(CH₂)ₙNR^{3D}R^{3E}, wherein n is 2 to 6 and of R^{3C}, R^{3D} and R^{3E} is, independently, H, a substituted or unsubstituted C₁₋₆ alkyl, substituted or unsubstituted C₁₋₄ alkaryl, or substituted or unsubstituted C₆₋₁₀ aryl, and with the proviso that at least one of R^{3α} and R^{3β} is not H;
R⁶ is CH₃, CH₂OR^{6A}, or CH₂OCOR^{6A}, where R^{6A} is H, substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl;
R¹⁴ is OH, Cl, OR^{14A}, or OC(O)R^{14A}, where R^{14A} is a substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl, or R¹⁴, R^{15β}, and the carbons they are bonded to together represent an epoxide;
each of R^{15α} and R^{15β} is, independently, H, OH, OR^{15A}, or OC(O)R^{15A}, where R^{15A} is a substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl, or R^{15a} and R¹⁵ together are ═O;
each of R^{16α} and R^{16β} is, independently, H, OH, OR^{16A,} or OC(O)R^{16A}, where R^{16A} is a substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl, or R^{16α} and R¹⁶ together are ═O;
R¹⁷ is 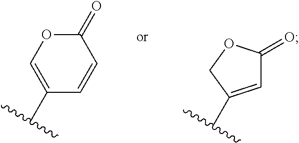
R¹⁸ is CH₃, CH₂OR^{18A}, or CH₂OCOR^{18A}, where R^{18A} is H, substituted or unsubstituted C₁₋₆ alkyl, substituted or unsubstituted C₁₋₄ alkaryl, substituted or unsubstituted C₆₋₁₀ aryl, substituted or unsubstituted C₁₋₄ alkheteroaryl, or substituted or unsubstituted C₁₋₉ heteroaryl;
with the provisos that no carbon atom that is bonded to OH is bonded to another group via an oxygen bond.

2. The method of claim 1, wherein said compound is selected from the group consisting of bufalin, 3α-hydroxybufalin, bufalin 3-acetate, bufalin 3-succinate, bufalin 3-methacrylate, bufalin 3-suberate, bufalin 3-methylsuberate, bufalin 3[N-(tert-butoxycarbonyl)hydrazido]succinate, 3-oxobufalin, 14α-hydroxybufalin 3β,16β-diacetate, scillarenin, 3-oxoscillarenin, bufotalin, desacetylbufotalin, gamabufotalin, gamabufotalin 3-acetate, 3-oxogamabufotalin 11-acetate, telocinobufagin, hellebrigenin, acetylarenobufagin, 15α-hydroxybufalin, 15α-hydroxybufalin 3-acetate, 15-oxobufalin 3-acetate, resibufagin, resibufaginol, resibufagenin, 3α-hydroxyresibufogenin, resibufagenin 3-acetate, 3-oxoresibufogenin, Δ¹-3-oxoresibufogenin, Δ^{1,4}-3-oxoresibufogenin, 16α-hydroxyresibufagenin 3-acetate, 14α,15α-epoxyresibufogenin, 3α-hydroxy-14α,15α-epoxyresibufogenin 3-acetate, 3-oxo-14α,15α-epoxyresibufogenin 3-acetate, 14α,15α-epoxyresibufogenin 3-acetate, 14α,15α-epoxyresibufogenin 3α-acetate, marinobufagin, periplogenin, digitoxigenin, digitoxigenin 3-acetate, digitoxigenin 3-suberate, digitoxigenin, 3-methylsuberate, Δ^{1,4}-digitoxigenin, cinobufagin, 3α-hydroxycinobufagin, cinobufagin 3-acetate, cinobufagin 3-succinate, cinobufagin 3-suberate, cinobufagin 3-cinnamate, 3-oxocinobufagin, cinobufagin 3,5-dinitrobenzoate, 3,16-diketocinobufagin, 16-oxocinobufagin 3-acetate, desacetylcinobufagin, desacetylcinobufagin 3-acetate, desacetylcinobufagin 3-acetate 16-succinate, desacetyl-14α,15α-cinobufagin 3-acetate, cinobufotalin, desacetylcinobufotalin, β-chlorohydrin, 14β-artebufogenin, 14β-artebufogenin 3-acetate, 14α-artebufogenin, 3-oxo-14α-artebufogenin, Δ^{1,4}-bufalin, Δ^{1,4}-3-oxobufalin, Δ^{1,4}-bufotalin 3-acetate, 7β-hydroxybufalin, 1β,7β-dihydroxybufalin, 16α-hydroxybufalin, 7β,16α-dihydroxybufalin, 3-epi-desacetylcinobufagin, 1β-hydroxy desacetylcinobufagin, 3-epi-desacetylcinobufotalin, cinobufagin 3-O-β-D-glucoside, 3-epi-7β-hydroxybufalin, telocinobufagin, 11β-hydroxybufalin, 15α-hydroxybufalin, 15β-hydroxybufalin, 12β-hydroxybufalin, 1β,12β-dihydroxybufalin, 12β-hydroxycinobufagin, 12β-hydroxy desacetylcinobufagin, 3-oxo-12β-hydroxycinobufagin, 3-oxo-12β-hydroxy desacetylcinobufagin, 12-oxo-cinobufagin, and 3-oxo-12α-hydroxycinobufagin.

3. The method of claim 1, wherein said compound is

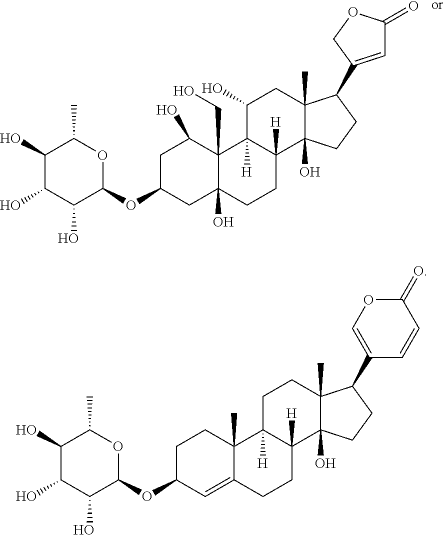

4. The method of claim 1, wherein R^{3α} and R^{3β} together are ═NNR^{3C}(CH₂)ₙNR^{3D}R^{3E} or ═NO(CH₂)ₙNR^{3D}R^{3E}, where n is 2 to 6 and each of R^{3C}, R^{3D} and R^{3E} is, independently, H, a substituted or unsubstituted C₁₋₆ alkyl, substituted or unsubstituted C₁₋₄ alkaryl, or substituted or unsubstituted C₆₋₁₀ aryl.

5. The method of claim 1, wherein said compound is ouabain or proscillaridin.

6. The method of claim 1, wherein said ocular disorder is selected from the group consisting of angiogenic ocular disease, ocular inflammation, retinopathy, retinopathy of prematurity, diabetic retinopathy, macular degeneration, age related macular degeneration, contact lens overwear, corneal graft rejection, corneal neovascularization, choroidal neovascularization, corneal graft neovascularization, retinal neovascularization, cortical visual impairment, epidemic keratocon junctivitis, marginal keratolysis, Mooren ulcer, myopia, pars planitis, phylectenulosis, post-laser surgery complications, pterygium, radial keratotomy, retrolental fibroplasias, ocular ischemic syndrome, retinal ischemia, ischemic optic neuropathy, non-arteritic ischemic optic neuropathy, glaucoma, neovascular glaucoma, hypoxia related ocular surface inflammation, ocular or macular edema, ocular neovascular disease, superior limbic keratitis, Steven Johnson disease, Terrien's marginal degeneration, scleritis, radial keratotomy, uveitis, vitritis, myopia, optic pits, chronic retinal detachment, post-laser treatment complications, cataracts, cataract surgery, conjunctivitis, Stargardt's disease, Eale's disease, central retinal vein occlusion, and sickle cell retinopathy.

7. The method of claim 1, wherein said ocular disorder is associated with a systemic hypoxic disorder selected from the group consisting of hypotension, diabetes, angiogenic disorders, cancer, autoimmune disease, inflammatory conditions, atherosclerosis, stenosis of the carotid artery, Vitamin A deficiency, Stargardts disease, Wegeners sarcoidosis, and age-related metabolic changes.

8. The method of claim 5, wherein said ocular disorder is selected from the group consisting of retinal neovascularization, choroidal neovascularization, corneal neovascularization, diabetic retinopathy, retinopathy of prematurity (ROP), macular degeneration, age-related macular degeneration (ARMD).

9. The method of claim 1, wherein said ocular disorder is characterized by. ischemia.

10. The method of claim 9, wherein said ocular disorder characterized by ischemia is selected from the group consisting of ocular ischemic syndrome, retinal ischemia, ischemic optic neuropathy, non-arteritic ischemic optic neuropathy, glaucoma, and neovascular glaucoma.

11. The method of claim 1, wherein said compound is formulated for ocular administration.

12. The method of claim 11, wherein said compounds is administered to the eye topically, by injection, or using an intraocular device.

13. The method of claim 11, wherein said compound is formulated for sustained release.

14. The method of claim 1, wherein said compound is administered in combination with an anti-VEGF therapeutic.

15. The method of claim 14, wherein said anti-VEGF therapeutic is and anti-VEGF antibody or a VEGF antagonist.

16. A method of treating or preventing an ocular disorder in a mammal mediated by HIF-1 that includes administering an effective amount of an agent to the mammal that antagonizes one or more elements of a pathway that leads to the endogenous biosynthesis of a cardiolide or bufadienolide.
